(19) United States
(12) Patent Application Publication
(10) Pub. No.: US 2008/0228408 A1
(43) Pub. Date: Sep. 18, 2008

(54) Method for Determining the State of an Ensemble of Cells and System for the Implementation of the Method

(75) Inventors: Claude Reiss, Chevrieres (FR); Christophe Reiss, Pierre Chatel (FR)

Correspondence Address: YOUNG & THOMPSON, 209 Madison Street, Suite 500, ALEXANDRIA, VA, 22314, US

(73) Assignee: VIGILENT TECHNOLOGIES, CHEVRIERES (FR)

(21) Appl. No.: 12/067,812

(22) PCT Filed: Sep. 22, 2006
(86) PCT No.: PCT/FR2006/050938

§ 371 Date: Mar. 24, 2008

(30) Foreign Application Priority Data
Sep. 23, 2005 (FR) 0552854

(51) Int. Cl. G01N 33/48

(52) U.S. Cl. 702/19

(57) ABSTRACT

The invention concerns a method for determining the condition of at least one culture of prokaryotic or eukaryotic cells defined in greater detail herein, including in particular one or more steps for simultaneously analyzing the condition of a large number of biological markers of interest. The inventive method, enables, in some cases, the cellular response to a plurality of modifications of environmental conditions to be simultaneously determined, including, for example, the simultaneous determination of the cellular response to various compounds to be tested.

### FIELD OF THE INVENTION

The present invention refers to the field of in vitro evaluation of the answers of a living organism to an event.

In particular, the invention refers to the field of in vitro evaluation of answers of prokaryotic or eukaryotic cells to an intracellular dysfunction or a change in their external environment.

In particular, the invention refers to the field of the determination of change(s) of phenotype of the cells after the incubation of the aforesaid cells with a compound of which the effects on the organism are to be determined.

### FORMER ART

In many situations, either in the field of academic research or in the field of industrial research in biotechnology, it is important to determine, under conditions given, the physiological state of prokaryotic or eukaryotic cells.

In general, the physiological state of prokaryotic or eukaryotic cells can be, at least on certain aspects, determined by qualitative and/or quantitative detection of one or more biological markers of interest possibly contained or possibly expressed by these cells.

As biological markers of interest usually used, one can in particular quote genes, the products of transcription of genes and proteins. One can also use other metabolites of the cell as biological markers, including the intracellular enzymes.

One knows, in the state of the art, of many processes allowing the evaluation, at least partial, of the phenotypical state of prokaryotic or eukaryotic cells, by qualitative and/or quantitative detection of a biological marker or a whole of biological markers.

For example, request PCT n^{o} WO 00/28091 describes processes of analysis of gene expression data which includes/understands stages of comparison of gene expression profiles making it possible to determine the evolution of these expression profiles with time.

One knows also processes allowing to evaluate the answer of cells incubated with various compounds, including with compounds having a potential interest in therapy or with compounds having potentially toxic properties for the organism.

Thus, the American patents n^{o} U.S. Pat. No. 6,300,078 and n^{o} U.S. Pat. No. 6,859,735 describe systems for identification of cellular components targets of a pharmaceutical composition including/understanding a stage of quantification of the RNA, cDNA or of proteins derived from cells of interest.

The same type of techniques of detection and/or quantification of cellular components was also used to carry out qualitative and/or quantitative profiles of these cellular components, in order to diagnose a disease, as described for example in the patent application American n^{o} US 2001/0018182 or in request PCT n^{o} WO 03/083140.

One also described the use of gene expression profiling in the determination of a compound cell toxicity, for example in the patent application American n^{o} US 2002/0192671.

Processing of data generated by marker analysis and detection techniques are also described in patent application American n^{o} US 2005/0042622 or in the patent application American n^{o} US 2005/0060100.

### SYNOPSIS OF THE INVENTION

The present invention covers a method to determine the state of at least one (prokaryotic or eukaryotic) cell culture, which is defined in detail in the present description and which includes/understands in particular one or more stages allowing the simultaneous analysis of the state of a great number of biological markers of interest.

As this will be detailed further in the description, the method described in the invention makes it possible, in certain cases, the simultaneous determination of cellular answer to a plurality of environmental changes, including, for example, the simultaneous determination of the cellular answer incubated with various compounds to be tested.

Moreover, the method according to the invention makes it possible, in certain cases, to carry out a complete test quickly, thanks to the implementation of an early interruption stage of the said method as soon as sufficient, although non-exhaustive information on the state of the tested cells was generated.

The invention also has as an aim the definition of systems for the implementation of the said method of determination of the state of at least one (prokaryotic or eukaryotic) cell culture.

### DESCRIPTION OF THE FIGURES

FIG. 1 is a general outline of a system of determination of the state of a cell culture according to the invention. This general outline also makes it possible to present the principal stages of the method of determination of the state of a cell culture according to the invention.

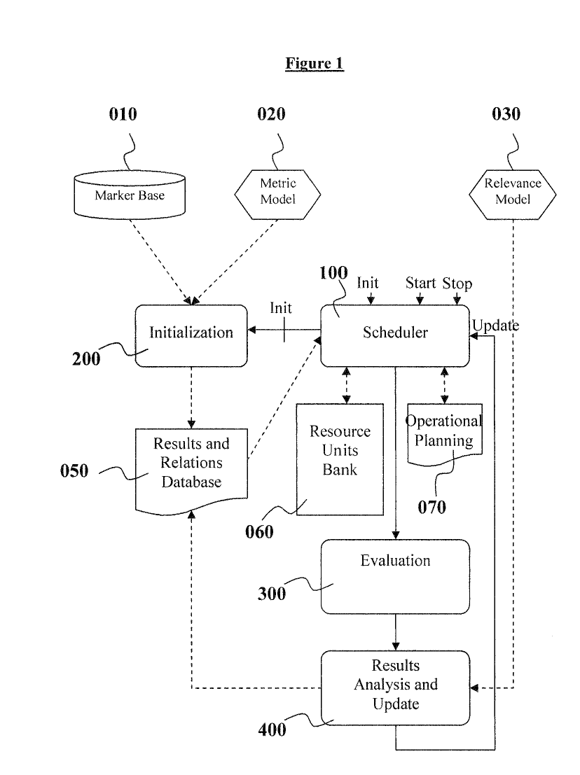

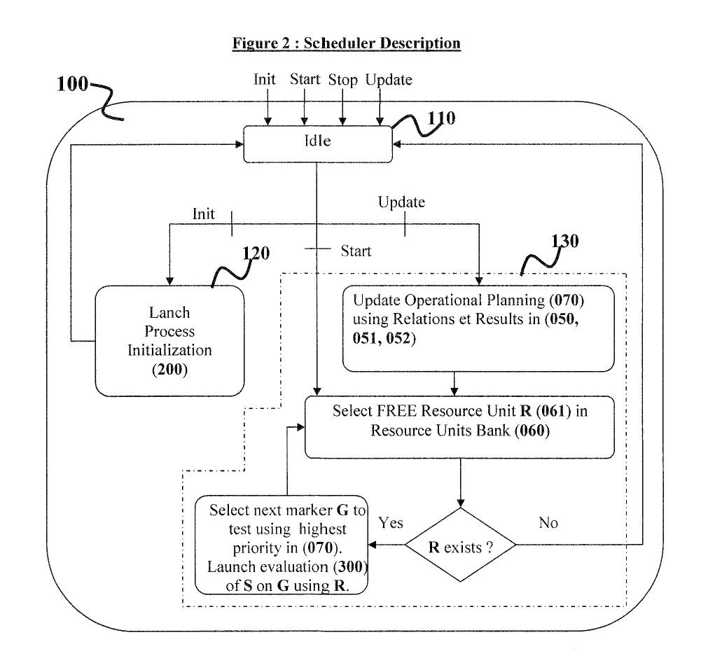

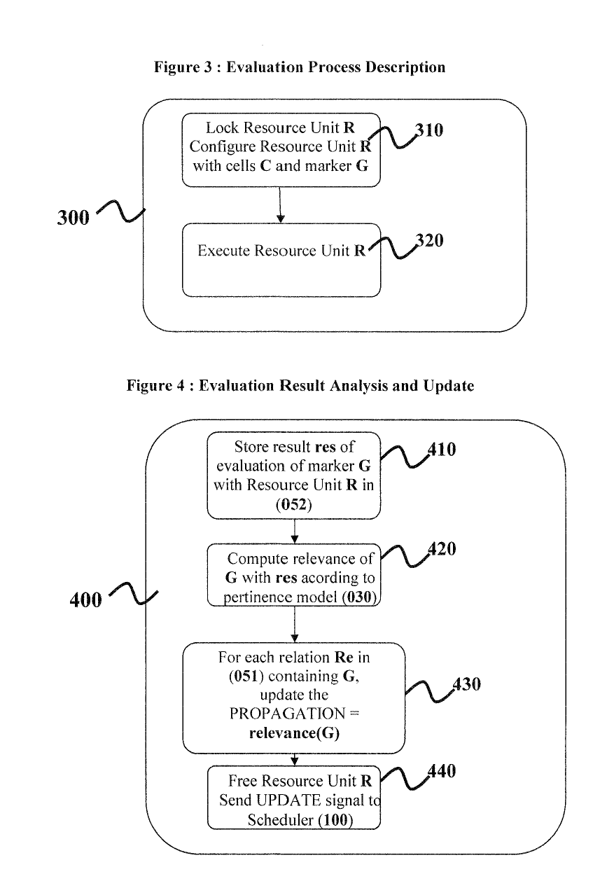

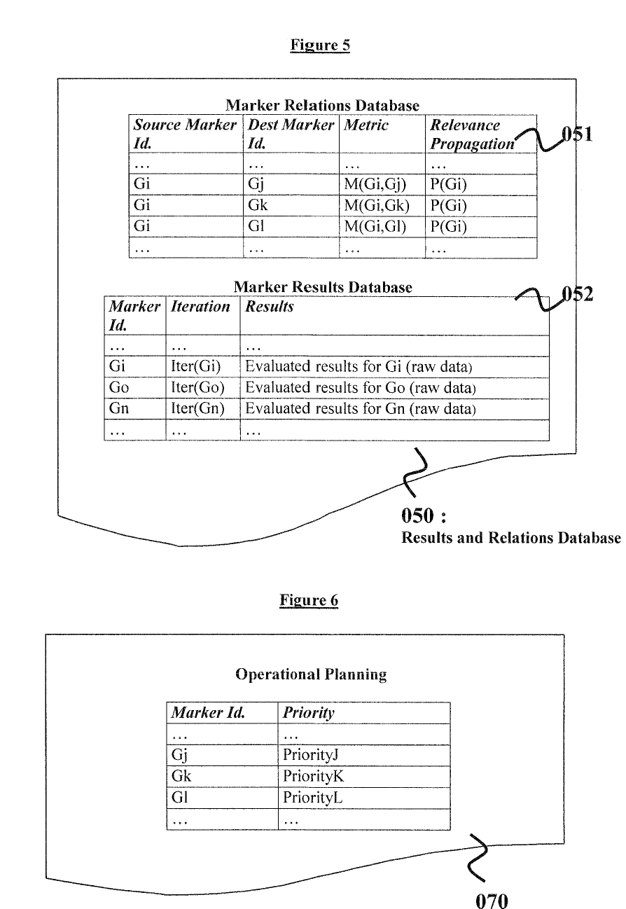

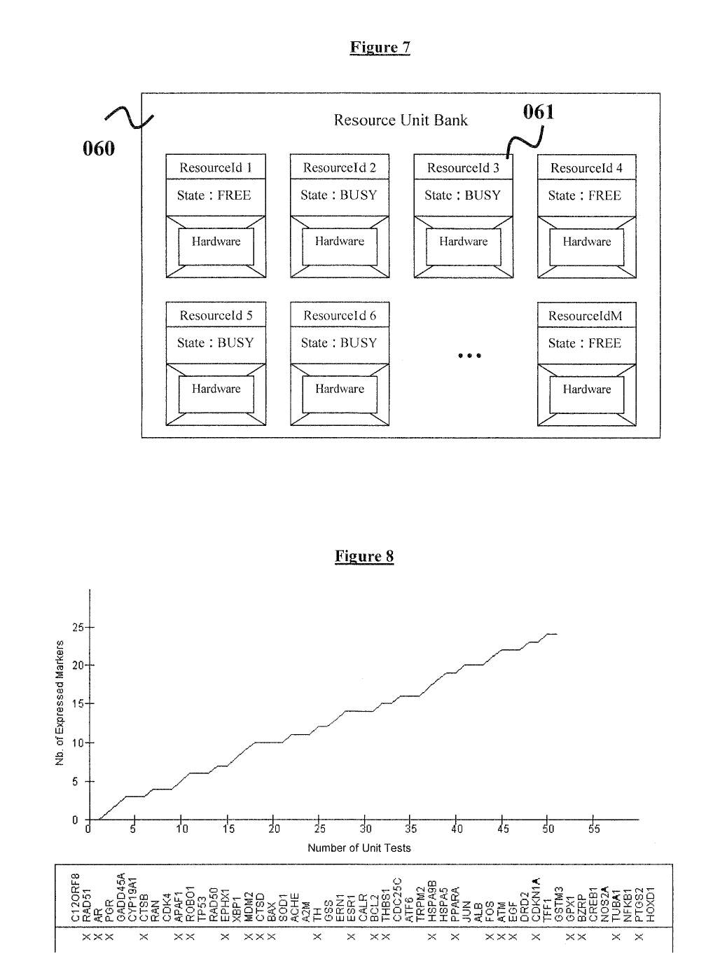

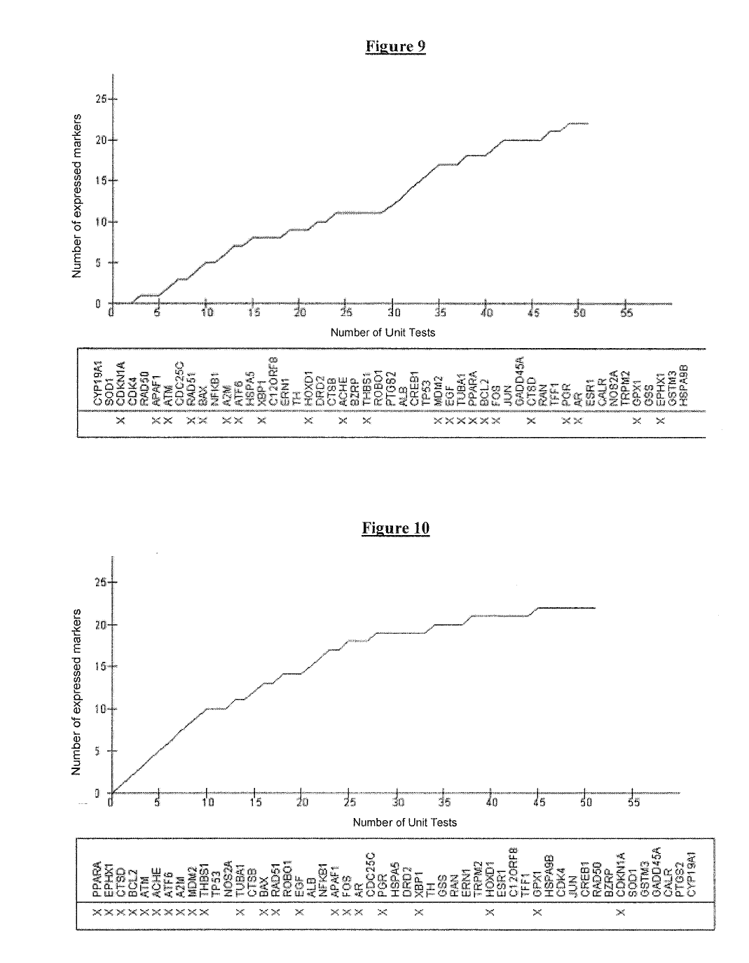

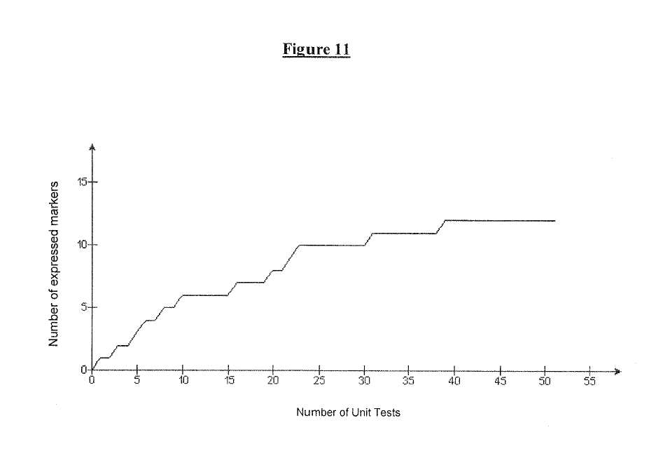

On FIG. 1, the sequence of the stages of the method, which uses successively the various means of the system, is represented by a succession of simple arrows.

On FIG. 1, the discontinuous arrows indicate the data transmission starting from means of information storage towards means of treatment. The discontinuous arrows are (I) mono-directional or (II) bidirectional, according to whether (I) data are transferred from a means of information storage towards a means of treatment, or contrary are transferred from a means of treatment towards a means of information storage, or (II) data can be indifferently transferred in a direction or the other, according to the needs necessary to the execution of the method.

FIG. 2 is a diagram representative of the sequence of operations of means known as Scheduler (100), during the execution of the method. The sequence of operation includes/understands in particular a sub-sequence 130, which orders the execution of the method during the steady phase which takes place between the starting and the ending of the method execution.

FIG. 3 is a diagram representative of the operation of the test execution means (300), during the execution of the method.

FIG. 4 is a diagram representative of the operation of the data computation and handling means (400), during the execution of the method.

FIG. 5 is a diagram representative of the possible structure of the data storage means, known as Results and Relations Database (050), which includes/understands a data storage means, known as Marker Relation Database (051) containing information on relation between biological markers and a data storage means, known as Marker Result Database (052) containing results of the tests carried out during the execution of the method and generated by the Resource Units (061). The content of means (050) is updated dynamically during the execution of the method. The means (050) constitutes a common total file usable by the various means of a system of determination according to the invention, for the execution of the stages of the method which require the access to information that the means (050) of storage contains.

FIG. 6 is representative of the possible structure of the means known as Operational Planning (070), during the execution of the method. The Operational Planning (070) contains data relative to the row of priority of the various biological markers not yet tested at a given moment, these data being used by the Control Unit (100) during the execution of the method to establish the chronology of the tests which remain to be possibly executed.

FIG. 7 is a diagram representative of the means known as the Unit Bank (060) that contains means known as Resource Units (061) used during the execution the method.

FIG. 8 is a diagram representative of the evolution of the execution of the method described in the invention; using a strategy of random selection of markers, implemented according to the invention, applied to the evaluation of Paraquat. In X-coordinates: number of execution steps of the method (=amount of unit tests actually executed). In ordinates: number of markers actually expressed (i.e. meaningful markers tested).

FIG. 9 is a diagram representative of the evolution of the execution of the method described in the invention; using a strategy of selection by class of markers, implemented according to the invention, applied to the evaluation of Paraquat. In X-coordinates: number of execution steps of the method (=amount of unit tests actually executed). In ordinates: number of markers actually expressed (i.e. meaningful markers tested).

FIG. 10 is a diagram representative of the evolution of the execution of the method described in the invention; using a strategy of self-adaptative selection of markers, implemented according to the method described in the invention, applied to the evaluation of Paraquat. In X-coordinates: number of execution steps of the method (=amount of unit tests actually executed). In ordinates: number of markers actually expressed (i.e. meaningful markers tested).

FIG. 11 is a diagram representative of the evolution of the execution of the method described in the invention; using a strategy of self-adaptative selection of markers coupled to a preliminary analysis by QSAR, implemented according to the method described in the invention, applied to the evaluation of Rotenone. In X-coordinates: number of execution steps of the method (=amount of unit tests actually executed). In ordinates: number of markers actually expressed (i.e. meaningful markers tested).

### DETAILED DESCRIPTION OF THE INVENTION

The applicant endeavoured to develop a method for the determination of the state of at least an ensemble of prokaryotic or eukaryotic cells which can be carried out with large scales, which are fast and inexpensive.

In particular, one sought the development of a method allowing simultaneously the test of the state of a great number of potentially relevant biological markers with respect to a given physiological situation. The method relies on the management in real-time of the number of biological markers to test (dynamic assignment of markers) on the one hand, and on the management in real-time of the order in which the various biological markers are tested (dynamic scheduling of markers) on the other hand.

The method according to the invention aims at determining the state of at least an ensemble of prokaryotic or eukaryotic cells by means of the determination of the state of an ensemble of biological markers contained or expressed by the aforementioned cells, the aforementioned method including the stages A, B, C, D, E, F described in the following, so that the stage A is reached only once at the beginning of the evaluation (initialization), stages (B, C, D, E) forming a sequence of operations carried out in a recurrent way as many times as necessary, and the stage F is reached only once at the end of the evaluation (termination).

Thus, it is an aim of the invention to provide a method for the determination of the state of at least an ensemble of prokaryotic or eukaryotic cells using an ensemble of relevant biological markers, including the following stages:
A) a stage of initialization during which (I) one loads one or more data storage means with (i) data identifying biological markers that are potentially relevant for the specific execution of the method, as well as (ii) data defining metric between biological markers (quantification of similarity between the markers), (iii) a numerical relevance model and (iv) initial priority data for all the biological markers used (these concepts being thereafter defined), and (II) one initializes devices used to test the state of the selected markers, the aforementioned devices consisting of “Resource Units”;
B) a stage of progression of the method during which the state of one or more biological markers are tested by one or more Resource Unit, such as biological markers to test at this stage are dynamically selected given the ranking of their priority at this time
C) a stage of recovery of the raw results produced by tests carried out by the Resource Units of at the stage B);
D) a stage of analysis of raw results recovered at the stage C), which includes in particular a calculation of relevance of each test which was carried out;
E) a stage of update of the initial data relative to each marker tested, according to the data obtained from the tests at stage D), the aforementioned stage also including an update of the priority of each marker not tested yet for the next execution of the cycle including the stages B) to E); and
F) a stage of termination of the system, reached when a condition of termination of the method occurs.

The information generated during the execution of the method according to the invention, along with the set of markers actually tested during the execution of the method according to the invention account for the state of the set of prokaryotic or eukaryotic cells.

The invention has as an aim the definition of a method to determine the state of at least an ensemble of prokaryotic or eukaryotic cells by means of the determination of the state of an ensemble of biological markers contained or expressed by the aforementioned cells, the aforementioned method being implemented by a system for the determination of the state of at least an ensemble of prokaryotic or eukaryotic cells, the aforementioned system including:
1) a data storage means known as Marker Base (010) including an ensemble of data characterizing a number Nm of biological markers G1;
2) a data storage means known as Metric Model (020) characterizing any relation Rm between two biological markers Gi among the Nm biological markers indexed in the means (010), the aforementioned means (020) containing for each relation between two biological markers present in (010):
   a reference of identification of a first biological marker G1 present in the means (010);
   a reference of identification of a second biological marker G2 present in the means (010);
   a numerical value METRIC(Rm) defining the metric of relation Rm between the first and the second marker;
3) a means (060-G) containing a number Nru of Resource Units (061), where each Resource Unit (061) is able to carry out and transmit at least one state parameter of a biological marker Gi, contained or expressed by a cell culture C, towards an evaluation means (300), each Resource Unit (061) including:
   an identification means (0611) of the aforesaid Resource Unit (061);
   a status means (0612) that indicates at any time the operating state of the aforesaid Resource Unit (061);
   a means (0613) of determination of at least one state parameter of a biological marker Gi, contained or expressed by a cell culture C;
   means (0614) of signal transmission between the aforementioned Resource Unit (061) and the evaluation means (300);
4) a data storage means known as Results and Relations Database (050) containing information about of biological markers Gi, contained or expressed by a cell culture C, the aforementioned means (050) including:
   a data storage means known as Marker Relation Database (051) containing information about relations between at least several biological markers selected in the group of the Nm biological markers indexed in the means (010), the aforementioned information consisting of, for each biological marker indexed in (010):
      a reference of identification of a first biological marker Gi present in the means (010);
      a reference of identification of a second biological marker present in the means (010);
      a value METRIC (Rm) defining the metric of the relation between the first and the second marker; and
      a value P(Gi) defining the relevance of the first marker in a test carried out for the given marker Gi and an ensemble of cell culture determined, the aforementioned value P(Gi) being calculated by an analysis means (400);
   a data storage means known as Marker Results Database (052) of containing results of tests carried out by one or more Resource Units (061) contained in the Resource Unit Bank (060), the aforementioned Marker Results Database (052) containing a set of test results, each test result containing:
      an identifier of a biological marker present in the means (010) that has already been tested by a Resource Unit (061) contained in (060), and for which at least one state parameter value has been measured;
      a ranking value specifying the position of test of the said biological marker in the evaluation in progress;
      at least one state parameter value defining the result of the test carried out with the aforementioned biological marker by the aforementioned Resource Unit (061).
5) an evaluation means (300) used to evaluate results of tests carried out by one or more Resource Units (061) included in the means (060), the aforementioned means (300) comprising:
   data storage means for at least one state parameter coupled to a biological marker Gi, the aforementioned state parameter being generated by a Resource Unit (061);
   means to receive and transmit a signal between the aforementioned means (300) and Resource Units (061) present in the means (060);
   means for ordering the execution of tests by one or more Resource Units (061);
   means to transmit a signal from the scheduler (100) towards the aforementioned means (300);
   means to transmit a signal from aforementioned means (300) towards a means (400) of analysis of the results of test.
6) a means known as Relevance Model (030) for the storage of a function of calculation of a value P(Gi) defining the relevance of a biological marker Gi indexed in the means (010);
7) an analysis means (400) for the calculation of the value P (Gi) defining the relevance of a biological marker Gi indexed in the means (010) for a given cell culture C; the computation using the function stored in the means (030) with at least one state parameter defining the result of the test carried out with the aforementioned biological marker Gi by a Resource Unit (061), the aforementioned state parameter being provided to the means (400) by the means (300), the aforementioned means (400) containing a means of reception of a signal transmitted by the means (300);
8) a means known as Operational Planning (070) including:
   a set of test execution priority data for the biological markers Gi, the aforementioned means (070) containing, at every time of the evaluation and for each biological marker (Gi) not yet tested:
      an identifier of the said biological marker Gi for identification in means (010); and
      a value PRIORITY defining the level of priority of the said biological marker Gi;
   means of transmission of signals between the aforementioned means (070) and the scheduler (100);
   means allowing the initial configuration of the priority data for each marker contained in (010); data of priority being numerical positive or null values
9) an Initialization means (200) of the system including:
   transmission means of a signal between the aforementioned means (200) and the method of control (100);
   transmission means of a signal between the aforementioned means (200) and the means (010) containing the set of data characterizing a number Nm of biological markers Gi;
   transmission means of a signal between the aforementioned means (200) and the Metric Model (020) characterizing a metric relation RM between two biological markers Gi;
   transmission means of a signal between the aforementioned means (200) and the Results and Relations Database (050) containing state parameters of biological markers contained or expressed by a cell culture C.
   transmission means of a signal between the aforementioned means (200) and the Operational Planning (070) containing a set of data characterizing the initial priorities of the Nm biological markers.
10) a method of control, known as Scheduler (100) including:
   transmission means of a signal between the aforementioned means (100) and each Resource Unit (061);
   transmission means of a signal between the aforementioned means (100) and the Operational Planning (070);
   transmission means of a signal between the aforementioned means (100) and the means (300) of analysis of the test results;
   transmission means of a signal between the aforementioned means (100) and the Results and Relations Database (050);
   means of controlling operations of the Resource Units (061),
      the aforementioned method including/understanding the following stages:
A) an initialization stage during which the method of control, known as Scheduler (100) carries out the following operations:
   a1) (I) to load in the means (051) the data contained in the means (010) and (020);
   (II) to load in the means (030) information and instructions concerning the relevance calculation function used for the Nm biological markers contained in the means (010);
   (III) to load in the means (070) information about initial priority, for at least part of the biological markers selected among the Nm biological markers of (010); and
   a2) to select, starting from an ensemble (060-G) of Resource Units (061), an ensemble, known as Resource Unit Bank (060) of Resource Units (061) designed to carry out the test of the state of at least part of the biological markers Gi loaded in stage a1) in the means (051) and (070), and initialize each Resource Unit (061) of the means (060);
B) start the system so that the method of control (100) carries out the following operations:
   b1) select in the means (070) the biological marker Gi having the highest priority value PRIORITY, then remove the data of the said biological marker Gi of the contents of the means (070);
   b2) add, in the means known as Marker Results Database (052), the data associated to the said biological marker Gi, comprising:
      the identifier of the said biological marker Gi present in the means (051), and
      a value specifying the rank of test of the said biological marker Gi in the global evaluation.
   b3) select a Resource Unit (061) with internal state value “FREE”, designed to carry out the test of the state of the said biological marker Gi; then transmit to the means (300) a signal of execution of the test of state of the said selected biological marker Gi, the aforementioned test being carried out by the aforementioned Resource Unit (061) after configuration of this one with the said biological marker Gi, the aforementioned Resource Unit (061) being assigned a “BUSY” internal state value;
C) recover the results of the tests carried out at the stage B), according to the following steps:
   c1) load state parameters generated by each Resource Unit (061) selected and executed at the end of the stage B) in the means of storage included in the means (300) of evaluation;
   c2) transmit the state parameters loaded at step c1) towards the means of storage (052) and towards the means of analysis (400);
D) analyze the results of the tests recovered at the stage C), according to the following steps:
   d1) calculate, for each biological marker Gi for which at least one state parameter was transmitted to means of analysis (400), the value P (Gi) defining the relevance of the said biological marker Gi, using the function stored in the means known as Relevance Model (030) with the aforementioned state parameters defining the result of the test carried out with the aforementioned biological marker Gi by the Resource Unit (061);
   d2) propagate the value P(Gi) calculated at step d1) for the aforementioned biological marker Gi towards the Marker Relations Database (051), the propagation consisting in assigning to each relation between any biological markers Gj not tested yet and said biological marker Gi a value of propagation: PROPAGATION (Gj, Gi)=P (Gi) in the Marker Relation Database (051);
   d3) free Resource Unit (061) used for the test of aforementioned biological marker Gi by assigning to the aforementioned Resource Unit the internal state value “FREE”.
E) update the marker data contained in the system, according to the following steps:
   e1) carry out a new classification of biological markers not tested yet while assigning to each biological marker Gi not tested yet a value of weight Pi calculated as the result of the following equation (1):

Pi=ΣMETRIC(Gi,Gk)*PROPAGATION(Gi,Gk); 1<=k=N,
in which:
   Σ is the operator “addition”
   N is the number of markers present in the means (010);
   METRIC (Gi, Gk) is the value in (051) defining the metric relation between the marker Gi and the marker Gk;
   PROPAGATION (Gi, Gk) is the value in (051) propagated at an occurrence of the previous step d2) during the test of the marker Gk if this one were already tested; PROPAGATION (Gi, Gk)=0 if Gk were not tested yet; and
   e2) update PRIORITY values in Operational Planning (070) with the new classification induced by the values Pi of weight obtained at the step e1) for each biological marker Gi not yet tested.
      The sequence of stages B)C)D)E) being reiterated until the first occurrence of the one of the conditions F) occurs:
F) conditions of termination:
   (I) interruption of the system by the user;
   (II) the value of a state parameter generated for a biological marker Gi by a Resource Unit (061) was defined as a termination condition of the system;
   (III) the stages A) to E) of the method were carried out for all the Gi markers indexed in the means (010),
      given that the set of data contained in the Marker Results Database (052) at the end of the stage F) constitutes an interpretation of the state of the prokaryotic or eukaryotic cell culture which is determined by the method, and given that at the end of the stage F), the Marker Results Database (052) contains information concerning the chronological order in which biological markers were tested by the method.

By “prokayotic cells”, one understands according to the invention bacteria cells.

By “eukaryotic cells”, one understands according to the invention cells from animals or vegetables, including plants or algae, cells of mushrooms, including yeasts, and the cells of protists.

By a “set of cells”, one understands according to the invention a plurality of cells, including an set of cells cultured in vitro or an set of cells recovered beforehand. An ensemble of cells can be obtained by recovery from a tissue sample taken from a multicellular organism whether animal, including human, or vegetable. An ensemble of cells can also be recovered from a sample recovered from the environment, including a sample of soil or mud from the natural environment.

Moreover, in the case mentioned above, the “set of cells” can be known or suspected to have been in contact, with one or more arbitrary substances, under conditions of concentration and exposure durations defined or not.

By a “set of cells”, one understands cells among those described above which are under specified conditions. For example, if one attempts to test, with the method of the invention, the effects of a substance on a specified cell type such as human hepatocytes, then one preferentially carries out the method of the invention successively or simultaneously with the following cells sets:
a pilot cell set consisting of a culture of hepatocytes in the absence of the substance to be tested,
a first series of cell sets to be tested, each set consisting of a culture of hepatocytes incubated with a concentration C of the substance to be tested during an exposure time T1, each cell set of the said series being incubated with a distinct concentration C of the substance to test,
one second series of cell sets to be tested, each set consisting of a culture of hepatocytes incubated with a concentration C of the substance to be tested during an exposure time T2, each cell set of the said series being incubated with a distinct concentration C of the substance to test,
. . . / . . .
an N^{th} series of cell sets to be tested, each set consisting of a culture of hepatocytes incubated with a concentration C of the substance to be tested during an exposure time T_{N}, each cell set of the said series being incubated with a distinct concentration C of the substance to test.

With the method as defined above, a unique “cell set” is tested, i.e. in the illustrative example above, the pilot set or any of the sets incubated with a given concentration of the substance during a determined exposure time.

The method as defined above allows to test any of the “cell sets” mentioned above and to compare the results to those obtained with another reference “cell set”, defined as another “cell set” mentioned above. In this case, the usage of the reference “cell set” at each stage of the method is an intrinsic function of the Resource Units, which then realizes simultaneously a test of the “cell set” and a test of the reference cell set, and produced results characterizing the difference in behavior between the two sets.

In certain modes of realization of the method, “the cell set” tested is a culture of cells of a given type CELL incubated for an exposure time T with a final concentration C of a substance S.

However, as described later below, a method in which several cell sets are tested in parallel is also included in the invention, the possibility of testing in parallel a plurality of cell sets depending primarily on the capacity for treatment and analysis of the system used for the execution, in parallel, of the method of the invention. In this mode of realization, several methods including stages A) to F) above are simultaneously carried out, each method being carried out with one determined cell set.

By “biological marker” contained in, or expressed by, the cells, one understands according to the invention any parameter associated with the cell, whose presence or state can be identified. The biological marker can be “contained” in the cells when this marker is an intrinsic parameter which in general does not vary during the lifetime of the cell, such as for example of the sequences of genomic nucleic acid. The biological marker can be “expressed” by the cells when the state of the biological marker can change during the lifetime of the cell, as for instance the level of expression of certain genes or the level of activity of certain enzymes.

In certain modes of realization of the invention, a “biological marker”, in the sense of the present description, can consist of a combination of at least two distinct markers, the term marker having its conventional technical meaning. For example, a single “biological marker” within the meaning of present description can consist of a combination of at least two markers of the expression level of a gene. The state parameters of such “a biological marker” consist of the combination of the values of the state parameters of each conventional marker contributing to the “biological marker”. As an illustration of these particular modes of realization of the invention, the state parameter(s) of one “biological marker” can indicate values of expression of a combination of at least two distinct genes, or values of expression of at least two distinct proteins. In these particular modes of realization of a “biological marker” of the invention, there are not limits to the numbers of conventional markers included in a single “biological marker”, other than the practical limit of the number of conventional markers available at the time of the implementation of the method or system of the invention. Ultimately, a “biological marker” according to the invention, whose finality is to account for the state of a total cellular metabolism under determined conditions of cell culture, can consist of the combination of the totality of the conventional markers available. In the majority of the cases, however, a “biological marker” within the meaning of the invention, when it incorporates a combination of conventional markers, includes less than 100 conventional markers.

The “biological markers”, who can be also indicated as Gi markers in present description, include the Gi markers which include one marker or a combination of markers, among the following conventional markers:
the presence or the absence of a nucleic acid in the genome of the cells tested, including the presence or the absence of a polynucleotide coding a protein, or the presence or the absence of a regulatory polynucleotide of regulation of the expression of a gene;
the presence or the absence of polymorphic sites in the sequence of a genomic nucleic acid of the cells tested;
the presence or the absence of a specific allele in a polymorphic site contained in a genomic nucleic acid of the cells tested;
the presence or the absence of a transcription product of a gene contained in the genome of the cells tested, including a messenger RNA (mRNA) or a complementary DNA (cDNA);
the presence or the absence of a small interfering RNA of the RNAi type, of a small interfering RNA of the RNAsi type, of an interfering micro-RNA of the RNAmi type, of a small nucleolar RNA of the RNAsno type, of a small nuclear RNA of the RNsn type;
the level of transcription of a gene contained in the genome of the cells tested;
the presence or the absence of a protein coded by the genome of the cells tested, or the presence or the absence of a protein coded by an organism hosted in the cells tested, for example a viral or fungus organism;
the level of translation of a protein coded by the genome of the cells tested, or the level of translation of a protein coded by an organism hosted in the cells tested;
intracellular or extracellular amount of a protein coded by the genome of the cells tested, or the intracellular or extracellular quantity of a protein coded by an organism hosted in the cells tested;
the presence or the absence, or amount, of any other detectable cellular metabolite, including factors of transcription, epigenetic control factors, hormones, enzymes cofactors, cofactors of intracellular or extracellular receptors, lipids, fatty-acids, polyosides, vitamins or trace elements.

For the clarity of the presentation of the method or system according to the invention, a “biological marker” below will generally include only one conventional marker.

By “set of biological markers”, one in general understands a plurality of biological markers which can be of one or several of the biological marker types described above.

Preferentially, for the implementation of the method of the invention, one uses a biological markers set said “homogeneous”, i.e. an ensemble of markers containing biological markers belonging to only one type of markers, for example among the types of biological markers described above. On a purely illustrative basis, the set of biological markers can include exclusively markers of the type “level of transcription of a gene”. In this illustration, the tests providing the state parameter of each biological marker can be carried out on the same type of test device, for example DNA chips.

Preferentially, an ensemble of biological markers whose state is determined by the method above does not result from a purely arbitrary choice. Thus, for the implementation of the method to determine if the set of cells tested are in a given physiological state, one chooses an ensemble of biological markers among the biological markers whose state is likely to provide relevant information on the aforementioned physiological state.

For example, if the method is implemented in order to determine if a substance or a compound is toxic for the cells, the set of the biological markers which is selected includes at least one or more biological markers whose state, for example the presence, the absence or the quantity, is informative on the response of the cells to a toxic compound.

In a general way, when the biological markers are not selected in a purely arbitrary way, they are selected on the basis of their degree of informative relevance with respect to the physiological state of the cells which one seeks to determine. The degree of informative relevance of a biological marker with respect to a given physiological situation can be given starting from the knowledge about the aforementioned biological marker in the state of the art. The degree of informative relevance of a biological marker with respect to a given physiological situation can also be given on the basis of relevance data generated for this specific biological marker, during one or several former executions of the method of the invention performed with an ensemble of biological markers including this specific marker.

Another initial variable of the above method consists of the set of prokaryotic or eukaryotic cells which is tested. According to the question asked at the beginning of the method, one uses a cells set of a suitable type.

For example, when one attempts to determine, with the method of the invention, if a substance is neurotoxic, one preferentially carries out the aforementioned method with at least an ensemble of neuronal cells. In such a mode of realization, the method is preferably carried out with a “cells set” consisting of the combination of (I) a culture of neuronal cells and (II) a biological markers set whose state is informative of cytotoxicity, and better of neurotoxicity. Still better, in this particular mode of realization, one carries out the method successively or simultaneously with various types of cerebral cells, for example unipolar or bipolar neurons, astrocytes, glial cells or Schwann cells.

The method of the invention provides, after its completion at the stage F), a set of state parameters including the parameter(s) of given state(s) for at least part of the biological markers of the initially selected biological markers set, and more precisely a set of state parameters including the parameter(s) of given state(s) for all the biological markers tested before the completion of the method.

Moreover, the method of the invention provides, after its completion at the stage F), the chronological sequence of the use of the markers realized during the execution of the method for an ensemble of cells CELL1, so that the method of the invention makes it possible to re-use later on this chronological sequence within the framework of an other execution using the same markers on an arbitrary ensemble of cells CELL2 in order to be able to compare in real-time the progression of the evaluation of CELL2 compared to the results obtained for CELL1.

The set of state parameters for the tested biological markers which is provided at the completion of the method of the invention defines the state of the tested ensemble of cells, in a given environmental situation.

For example, if the set of state parameters which is provided at the completion of the method includes one or more state parameters of informative markers with respect to a cell situation of apoptosis, the execution of the method will have made it possible to detect a cellular situation of apoptosis, under the environmental conditions to which the ensemble of cells was exposed before the execution of the method. These environmental conditions to which the ensemble of cells was exposed before the execution of the method can be, for example, the incubation of the cells in the presence of a compound for which one seeks the harmful or beneficial effects for the cells.

On a purely illustrative basis, if the set of biological markers selected at the beginning of the method includes a marker reporting the production level of the protein p53 and if the set of state parameters provided after the completion of the method includes a state parameter meaning the intracellular presence of a great quantity of protein p53, then the set of state parameters provided after the completion of the method is informative on the existence of a physiological situation of apoptosis in the ensemble of cells tested in the method.

The example above illustrates the extreme case where a state parameter of a single biological marker among the set of the biological markers selected at the beginning of the method is informative by itself on the physiological situation of the cells tested.

Also, in particular modes of realization of the method, when one seeks to determine the existence of a given physiological situation of the cells tested, the biological set of markers selected at the beginning of the method can include at the same time one or more biological markers whose state parameters provided separately for each one is only indicative but not enough informative with respect to the aforementioned physiological situation; in this case it is only the combination of several state parameters of markers that is informative, with respect to the aforementioned physiological situation to be determined.

In general, the physiological situation of the set of cells tested is defined by a combination of several state parameters of biological markers included in the set of state parameters provided after the completion of the method. For example, one can determine the stimulation of a particular metabolic pathway, when a combination of parameters of state, included in the set of state parameters provided after the completion of the method, indicates, independently or simultaneously.
an high expression level of genes coding for one or several proteins implied in the aforesaid metabolic pathway;
a great quantity of proteins implied in the operation of the aforesaid metabolic pathway;
an high level of enzymatic activity, for example when the proteins implied in the aforementioned metabolic pathway include one or several enzymes.

In some cases, a given biological marker can be informative for various distinct physiological situations.

As defined above, the method of the invention is carried out using various means, which will be specified below when necessary, before describing in detail the various stages of the method.

For the clarity of the description of the system and of the method of the invention, those means are described below in reference to the particular mode of realization described in FIGS. 1 to 7. In FIGS. 1 to 7, the various means can consist of physical or logical means of execution of the various stages of the method of the invention.

The method of the invention, which was defined in a general way above, is described below with its various specific characteristics and its preferred modes of realization.

For the sake of comprehension, the system and the method are described below for an execution of the method with only one set of cells present in a cell culture CELL.

### Stage A) of the Method

The stage A) consists of an initialization stage of the system during which data concerning biological markers are transferred from a means of global data storage (010) towards a means of storage (050) used for a cycle of executions of the method (stage a1)); the data concerning initial priorities of biological markers used are loaded in the means (070); the data defining the function of calculation of relevance for the biological markers used are transferred in the means (030); and the test devices test, called “Resource Units” are also initialized (stage a2)).

The method of the invention is executed with at least a subset of the biological markers who are indexed in the means (010), as represented on FIG. 1.

### The Means (010) of Storage

The means (010) includes a set of data characterizing a number Nm of biological markers. The number Nm of biological markers indexed in the means (010) is truly limited only by storage capacities and/or by treatment capacity of the said system, and also by the absolute number of biological markers that are available during the execution of the method, when storage capacities and/or of treatment capacity of the said system make it possible to index the totality of the biological markers known or available.

However, a set of limited size of biological markers is in general sufficient to carry out the method in multiple applications.

Thus, in general, number Nm of biological markers Gi indexed in the means (010) is at most 30.000, and often at most 10.000.

In many modes of realization of the invention, one selects at the stage a) at most 1000 biological markers among those which are indexed in the means (010).

In certain applications of the method, only the test of a small number of biological markers is necessary. In such applications, less than 100 biological markers can be selected at the stage a), among those which are indexed in the means (010).

The means (010) can include exclusively, for each indexed biological marker, a reference of identification of the said biological marker.

As indicated previously, the method of the invention is carried out for a combination (I) of a set of biological markers and (II) of an ensemble of cells.

Depending on the cellular type or the state of differentiation of the set of cells, one selects advantageously a set of biological markers Gi indexed in the means (010) likely to be expressed or to be informative for the type of cells used.

The Means (020) of Store

The means (020), known as Metric Model and loaded at the stage a) of the method consists of a means of storage for data specifying relations between each possible pair of biological markers, among the biological markers referred in the means (010) of storage and who are selected at the stage a).

The means (020) of storage can include a maximum number of relations between pairs of markers which follows the following equation (2):

Nb_relations=Nm·(Nm−1)/2 (2),

in which:
Nb_relations is an integer representing the maximum number of relations between markers that can be configured in the means (020); and
Nm is the total number of biological markers indexed in the means (010) of storage.

Each relation Rm between two given markers G1 and G2 among the Nm markers referred in the means (010), consists of a numerical value characterizing the distance between the two said markers, also called “metric” value, or METRIC (Rm), for the purpose of this description.

The value of METRIC (Rm) between two markers G1 and G2 is all the more large as the two markers G1 and G2 are close from each other, from an informative point of view, in a given metabolic context.

The value of METRIC (Rm) between two markers G1 and G2 contained in a means (010) depends on the informative context of the evaluation, so that two distinct evaluations using the markers G1 and G2 at different ends can use different values for METRIC (Rm).

On a purely illustrative basis, in a context of situation of inflammation, (I) a marker G1 representative of the level of expression of the gene of IL-1 and (II) a marker G2 representative of the level of expression of the gene of TNFa will share a large value of METRIC (Rm), since a high level of expression of each one of these two genes is physiologically associated with an inflammatory reaction of the organism.

Also on a purely illustrative basis, in a context of situation of inflammation, (I) a marker G1 representative of the level of expression of the gene of IL-1 and (II) a marker G2 representative of the level of expression of the gene of IL-2 will share a small value of METRIC (Rm), since the level of expression of gene of the IL-2 alone is not associated with an inflammatory reaction.

The METRIC value characterizing the metric relation between two markers is expressed in arbitrary units.

The METRIC value characterizing the metric relation between two markers G1 and G2 is null if the two markers do not have a common informative value, in a given informative context.

On a purely illustrative basis, in a context of situation of inflammation, (I) a marker G1 representative of the level of expression of the gene of IL-1 and (II) a marker G2 representative of the level of expression of the gene of the actine will have a value of METRIC (RM) equal to zero, since the level of expression of the gene of the actine is independent of the occurrence of an inflammatory reaction.

Given three distinct biological markers G1, G2 and G3, G1 is more similar to G2 than to G3 when the value of METRIC (G1, G2) is higher than the value of METRIC (G1, G3).

Before the first initialization of the system, one stores in the means (020) the metric data of relations for at least part of Nb_relations pairs of markers in (010). For example, values METRIC (Rm) of for each relations are calculated from the information known in the state of the art concerning a plurality of pairs of markers among Nb_relations possible pairs of markers.

At the first initialization of the system, the means (020) does not necessarily contain metric data for all possible Nb_relations pairs of markers. For the pairs of markers for which the means (020) does not contain metric data, the system allots a default null metric value (zero). At the first initialization of the system, the means (020) generally contains relation data for only a subset of the possible pairs of markers. Then, with the successive executions of the method, information about relations between pairs of markers is augmented, thanks to the accumulation of results from tests concerning markers involved in pairs for which no data of relation was initially present in the means (020).

### The Means (030) of Storage

The means (030), known as Relevance Model, contains necessary information (description, instructions) for the definition and implementation of an arbitrary function used to calculate the level of relevance suitable for the execution of the system in progress. The function of calculation will be applied thereafter during the execution of the system in progress to any result of test concerning any Gi marker loaded at the stage A) and contained in the means (051).

The means (070) of Operational Plan The means (070), known as Operational Planning, is essential for the execution of the method. The means (070) contains, for each marker Gi loaded at the stage A) and contained in the means of storage (051), at least one identifier of the said marker Gi and a value of PRIORITY, positive or null, for the aforementioned marker Gi. The means (070) contains, at the initialization of an execution of the system, preset values for each marker Gi, the values being positive, null or infinite positive (+∞). The system can in certain cases dynamically modify the contents of the means (070) during the execution in progress, as the results of test of the markers are known, as presented below. In certain modes of realization of the method, the initial values of PRIORITY are null for all markers. In some other modes of realization, the initial values of PRIORITY, for at least some of the markers, called “seeds”, are positive or are equal to (+∞). In this case, the initial priorities of the markers other that seeds are null. The means (070) thus contains at the stage a) the seeds suitable for the execution of the system.

As mentioned previously, the system and the process are described below for an execution of the method with only one cell culture, but can be easily extended to an execution of the method with more than one cell culture.

At stage A), the initialization of the system is ordered by the Scheduler (100) and is carried out by the means of initialization (200), which in turn orders the execution respectively of the stages a1) and a2). The stages a1) and a2) can be realized simultaneously or successively. The order in which the stages a1) and a2) are carried out is indifferent.

At the stage a1), the means (200) orders the loading, in the Marker Relation Database (051), of data contained initially in the means (010) (marker identifiers) and (020) (metric values for markers pairs), for at least part of the biological markers indexed or present respectively in the means (010) and (020).

On a purely illustrative basis, if the execution of the method aims at obtaining a set of state parameters for biological markers likely to be informative (I) with respect to a situation of cancer, or (II) with respect to a situation of inflammation, one realizes at the stage a1):
either a loading in the means (051) of markers likely to be informative with respect to a situation of cancer,
or a loading in the means (051) of the markers likely to be informative with respect to a situation of inflammation, respectively.

As represented on FIG. 5, the means (051) of storage includes/understands at least, for each biological marker Gi selected at the stage a1), a list of entries where each entry contains:
a reference of identification of the said marker Gi;
a reference of identification of another marker Gj;
for the pair of markers above, a value METRIC (Rm) defining metric relation between the two markers constitutive of the pair, when value METRIC (Rm) is known. If value METRIC (Rm) for the given pair of markers is not known, this value is set to NULL (0) by default in the means (051). On FIG. 5, value METRIC (Gi, Gj) between two markers Gi and Gj are indicated “M (Gi, Gj)”.
for the pair of markers above, a value of relevance PROPAGATION which is an informative value of the aforesaid the pair of markers.

At the stage a1), one assigns a null value (equal to zero) for all the values PROPAGATION contained in the means (051) of storage.

At the stage a1), the means (052) of storage of the results is empty.

At the stage a2) of the method, one selects a set (060) of Resource Units (061) among the whole of the Resource Units (061) present in the system, then one initializes these Resource Units (061). The system relies on a global set (060-G) of Resources Units (061). However, during the implementation of the method for the determination of the state of a given cell culture, it may not be necessary to test the totality of the biological markers available in (010) with to the totality of the Resource Units (061) present in (060-G).

At the stage a2) of the method, one selects only the Resource Units (061) that can carry out the test of state for biological markers selected at the stage a1). Thus, at the stage a2) one selects a set (060) of Resource Units (061) to test markers selected at the stage a1), this set (060) of Resource Units (061) consists of a sub-assembly of the total set (060-G) of Resource Units (061) that can be used for the realization of an of an execution of the method according to the invention.

At the stage a2), Resource Units (061) of the set (060) are re-initialized, All Resource Units (061) have an internal status set to “FREE”.

A set (060) of Resource Units (061) is represented on FIG. 7.

By “Resource Unit”, one understands according to the invention an unspecified device which is adapted to determine a state parameter of a biological marker.

A Resource Unit (061) consists of a logical unit which includes a physical device of test. The device of test can consist of a combination of elementary components which are associated. A Resource Unit can consist of a logical assembly of several elementary components. It is the aforementioned assembly, with its combination of components, which is adapted to the execution of a test of determination of a parameter of state of a biological marker Gi.

In certain modes of realization, a given device can be used as an elementary component commonly in several distinct Resource Units.

The Resource Units are generic in the sense that they are not a priori coupled with a given marker nor with a cell culture. On the contrary, Resource Units can be configured on demand with a given marker G and a given cell culture CELL, to perform a measure. This specialization is called in what follows the configuration of the Resource Unit with the marker G and the cell culture CELL. Once a Resource Unit was configured, it can be executed, i.e. it will carry out the operation of evaluation of one or more state parameters of said marker G in said cell culture CELL. Tests results are obtained by extracting the state and the values from Resource Unit's internal components. A Resource Unit can be re-initialized, i.e. that it returns in its initial state (recycling) where no cell culture and no marker are configured. Thus, after an operation of re-initialization of the Resource Unit, the Resource Unit can be re-used to carry out another unit test.

As that was already mentioned previously, a Resource Unit contains at least:
a means (0611) of identification of the aforesaid Resource Unit (061), which is indicated as “RessourceId” on FIG. 7;
a means (0612) of indication of the operating condition, at a given moment, of the aforesaid Resource Unit (061), which is indicated “Status” on FIG. 7. The means (0612) can take either the “BUSY” value, or the “FREE” value;
a means (0613) of determination of at least one state parameter of a biological marker G, contained or expressed by a culture of cells C;
means (0614) of transmission of at least one signal between the aforementioned Resource Unit (061) and the method of control, known as Scheduler (100);

A snapshot of a set (060) of Resource Units (061) is schematized on FIG. 7. On FIG. 7, the set (060) includes a number M of Resource Units (061) identified uniquely from “RessourceId1” to “RessourceIdM”. At the time the snapshot was taken, Resource Units “RessourceId1”, “RessourceId4” and “RessourceIdM” are in a “FREE” state, and Resource Units “RessourceId2”, “RessourceId3”, “RessourceId5” and “RessourceId6” are in “BUSY” state.

In many cases, a single Resource Unit (061) is able to determine a state parameter of a class of biological marker, that can cover a plurality of biological markers Gi indexed in the means (051).

The Resource Units (061) may be any device able to carry out the detection, the identification, and also possibly the quantification, the simultaneous presence in the cells or their environment of culture, of a substance or a plurality of substances implied in the cellular metabolism (anabolism or catabolism). The Resource Units (061) consists of devices suitable for the detection and/or the quantification of biological markers by, among others, genomic analysis, epigenomic, proteomic, metabolomic or glycomic.

For example, the state parameters of biological markers that one wants to determine can consist of the level of expression of a plurality of distinct genes. In this case, a Resource Unit (061) can consist of a set of automated means allowing the handling of one or more DNA chips on which are immobilized a plurality of distinct nucleic probes, allowing hybridization respectively with the various mRNA or with different cDNA corresponding to the products of transcription of the aforesaid distinct genes.

In this illustrative example, only one Resource Unit (061) can allow the determination of state parameters of the totality of the biological markers Gi indexed in the means (051).

Always in this illustrative example, the initialization of the Resource Units (061) includes the following operations:
(I) immobilize on an adapted support a plurality of nucleic probes allowing specifically the hybridization to the products of transcription of the various genes, whose level of expression is sought, then
(II) put in contact a biological sample, for example a cellular extract of mRNA or a sample of cDNA with the DNA chip prepared at the step (I) above.

The execution of the Resource Units consists in collecting, after an arbitrary duration of exposure of the chip with the biological sample, the values provided by the chip. The re-initialization of the Resource Unit consists of the cleaning of its components, or of their replacement, using automated means.

In certain modes of realization of the method, the various Resource Units (061) can be of distinct types. For example, the selected set (060) of Resource Units can include at the same time Resource Units (061) using DNA chips and Resource Units (061) using proteins chips.

### Stage B) of the Method

The stage B) is responsible for starting system and for monitoring its progression. In stage B), biological markers selected at the stage A), will be successively tested by means of the Resource Units (061).

At the beginning of the stage b1) of the method, one selects the biological marker Gi having the highest value PRIORITY in the means Operational Planning (070). The Operational Planning (070) is referred on FIG. 1. An example of contents of the Operational Planning (070) is represented on FIG. 6.

At the first initialization of the system, the Operational Planning (070) contains, for each marker Gi present in the means (051), at least (I) a reference of identification of the said marker Gi, along with a numerical value of priority that can be positive or null or infinite positive, arbitrarily fixed as the initial priority of markers. In the Operational Planning (070), biological markers are thus classified by decreasing order of priority value PRIORITY.

At step b1), one selects in the means (070) the references of identification of the biological marker Gi having the highest value of priority PRIORITY, then one removes the data of the said marker Gi of the contents of the means (070). If several markers share the same highest value of priority in (070), the selection between these markers is done in a random way.

The references of identification of the marker Gi which has just been selected at step b1) are added, at step b2) of the method, in the means known as Marker Results Database (052), as that is represented on FIG. 5.

At the step b2), one also adds in the means (052) an integer value ITERATION specifying the rank of the test of the said biological marker Gi in the evaluation in progress. The rank of test of a marker Gi, “Iter (Gi)” on FIG. 5, is expressed in arbitrary units. On a purely illustrative basis, the row of test of a Gi marker can consist of the date and the hour to which the test of determination of a parameter of state was carried out for the aforementioned Gi marker.

Then, at the step b3), one holds in (060) a Resource Unit (061) whose state (0612) is “FREE”, then one configures the said Resource Unit (061) with the aforementioned biological marker Gi. If all Resource Units contained in (060) are in “BUSY” state, it is waited until a Resource Unit is freed to carry out the reservation and the configuration. Then, a request for execution of the test of the state of the said selected marker Gi is transmitted to the means (300), the aforementioned test being carried out by the aforementioned Resource Unit (061), which is in turn set to “BUSY”. The means (300) for the execution of the step b3) is schematized on FIG. 3.

Thus, with the first execution of the step b3) in stage B), a first Resource Unit (061) is selected in set (060), thanks to a command addressed by the means (300) of evaluation, the means (300) of evaluation being itself under the control of the Scheduler (100).

Once the Resource Unit (061) was selected, its internal state is set to “BUSY”. This Resource Unit (061) cannot be selected until the test it carries out is completed.

Steps b1), b2) and b3) above are reiterated successively at stage B) for the markers having the highest priority in Operational Planning (070), this until the totality of the Resource Units (061) contained in the set (060) of Resource Units selected at the stage A) are in the state “BUSY”.

After execution, each Resource Unit (061) which carried out the totality of the test for the configured marker will come back to the inactive state and will be assigned a “FREE” state. Resource Units (061) in a “FREE” state are likely to be selected again, in a further given cycle of execution of the step b3), to carry out a test of parameter of state for another marker.

For example, a Resource Unit (061) consisting in a DNA chip can be selected a first time, at the step b3), to carry out a test of determination of the level of expression of a first gene (Gx marker). At the end of execution of the test for this first gene, the aforementioned Resource Unit (061) is again assigned of a “FREE” state. This Resource Unit (061) is then likely to be again selected during a later execution of the step b3), to carry out a test of determination of the level of expression of a second gene (Gy marker). Thus, a single Resource Unit (061) can be selected a plurality of time in the various execution of stage B) in the execution of the method, according to its availability and its capacity to carry out the test for a given marker at the time B) is executed.

In other modes of realization of the method, one selects as many Resource Units (061) that there are markers initially indexed in the means (051).

In the modes of realization of the method in which one seeks to evaluate the effects of a given substance S on a cell culture CELL using Nm biological markers Gi, one selects at the stage a) a set (060) of Resource Units (061), which can also be indicated “pool of resources”, containing a maximum number Nm of Resource Units (061).

As that will be exposed further in description, in certain modes of realization of the method, one can dynamically adjust the number of Resource Units (061) present in the set (060) of Resource Units which had been initially selected at the stage A) of the method. Thus, in certain modes of realization of the method, one or more Resource Units (061) can be dynamically added to the set (060) or on the contrary removed from the set (060), during the execution of the method.

### Stage C) of the Method

At the stage C) of the method, the results of each test carried out at the stage B) are recovered in order to be analyzed by the system. At the step c1), the state parameters of a marker Gi tested at the stage B) and which were generated by the responsible Resource Unit are loaded in a means of storage which is included in the means (300) of evaluation of the tests.

Then, at step c2), the state parameters of the marker Gi which were beforehand loaded in the means (300), are respectively transmitted towards the Marker Results Database (052) through the means of analysis (400).

By “state parameter” of a marker Gi, one understands according to the invention any information relating to the aforementioned marker produced by the Resource Unit which carried out the aforementioned marker's test. A state parameter of a marker Gi includes (I) the presence or the absence of the marker Gi in the sample tested, (II) the quantity of the marker Gi in the sample tested or (III) the physicochemical properties of the said marker present in the test. In certain modes of realization of the method, a given Resource Unit (061) can generate, at the stage C), several state parameters for a single marker Gi. Thus, for example, in the case of a Resource Unit (061) consisting of a DNA chip on which are immobilized nucleic probes specifically selected for their ability of hybridization with one or more transcription products of a given gene, the aforementioned Resource Unit (061) can generate two parameters of state for the same Gi marker, respectively (I) the presence or the absence of the products of transcription of said gene in the tested sample and (II) the quantity or the concentration of the products of transcription of said gene in the tested sample.

An illustration of a mode of realization of the Marker Results Database (052) is represented on FIG. 5.

At initialization of the system, the means (052) does not contain any data.

Then, with the successive executions of the cycle of steps b1), b2) and b3), the data (identifiers and test rankings) associated with the Gi markers successively selected at the stage b1) are added to the means (052) at the step b2). Thus, for each Gi marker indexed in the means (052), the aforementioned means (052) contains the reference of identification of the known as Gi marker and a value specifying the rank of test of said marker Gi, the value of order being referred, on FIG. 5, “Iter (Gi)”. The rank of test of a marker Gi, “Iter (Gi)” is expressed in arbitrary units. On a purely illustrative basis, the row of test of a Gi marker can consist of the date and the hour to which the test of determination of a parameter of state was carried out for the aforementioned Gi marker.

Then, with the successive executions of the cycle of stages b1), b2) and b3), the state parameters of the tested markers Gi which are generated by the Resource Units (061) are added at step c2), for each marker Gi, in the means (052) of storage, via the means (400) of evaluation.

Thus, at the end of the stage C), the means (052) contains at least a value of state parameter, for each Gi marker present in the aforementioned means (052), except if the test of this marker failed and if, for this marker, no state parameter was generated by the corresponding Resource Unit (061).

As that will be described later on in the present description, the method can be stopped before the complete execution of the cycles of steps b1), b2) and b3) for the totality of the Gi markers selected at the stage A), for example following a user request for a premature abortion of the method, or following the presence of a condition allowing the automatic abortion of the method.

In the modes of realization in which the method is stopped before the complete execution of the cycles of stages b1), b2) and b3) for the totality of markers selected at stage A), only part of the Gi markers indexed in the means (051) are also indexed in the means (052).

### Stage D) of the Method

The stage D) consists of a stage of analysis and quantification of the results for each test recovered at the stage C). After each execution of a Resource Unit for a given biological marker Gi of (051), at step d1) one calculates with the state parameters of the said biological marker Gi, transmitted towards means of analysis (400) at stage C), the value P (Gi) defining the relevance of the said biological marker Gi, by using the function stored in the means (030) with said the state parameters defining the result of the test carried out with the aforementioned biological marker Gi by a Resource Unit (061);

Then, at the step d2), one transmits the value P (Gi) calculated at step d1) for the aforementioned biological marker Gi towards the means (051) of storage;

Then, at step d3), one re-initialized the Resource Unit (061) used for the aforementioned biological marker Gi by assigning to the aforementioned Resource Unit the state value of “FREE”.

At the step d1), the raw results of the tests carried out at the stage B) and previously transmitted towards the Marker Results Database (052) via the means of evaluation (300) at step c2) are analyzed by the means of analysis (400).

The means of analysis (400) includes means of calculation of relevance of the raw results stored in the means (052). Calculations of relevance are carried out in the means (400) using instructions for the implementation of one or more algorithms which are initially contained in a means known as Relevance Model (030). The Relevance Model also indicates, for purposes of this description, the series of instructions of defining an algorithm or the combination of algorithms used to carry out the calculation of relevance of the raw results provided by state parameters of tested markers Gi.

The Model of Relevance is introduced into the means (030) system before the initialization at stage A) of the execution of the method.

On a purely illustrative basis, in certain modes of realization of the method for which the markers Gi consist of the level of expression of various genes, the Relevance Model consists of a function whose arguments are the level of expression of gene corresponding to each marker Gi.

Thanks to the definition of a Relevance Model stored in the means (030) and used by the means (400), the user can, with the execution of the step d1) of the method, have a value P (Gi) for each marker Gi tested before.

Preferentially, for the establishment of the Relevance Model stored in the means (030) before the initialization of an execution of the method, the following conditions must be followed.
(I) being given an ensemble of cells CELL; and
(II) being given the execution of a Resource Unit “RessourceId1” configured with a marker G1 and the sample derived from CELL;
(III) then:
   Relevance (G1)=0 if the execution of the Resource Unit “RessourceId1” does not generate any significant result, in the context of the test of state of the set of cells which is the subject of the execution of the method
   If not, Relevance (Gi)>0

According to the method, a phase of Propagation follows the phase of calculation of the values of Relevance.

The value of relevance P (Gi) of a given marker determined at the stage D) using of one or more state parameters generated for the aforementioned marker Gi at the stage B), is then used in the means (400) as indicated above. The value of relevance P (Gi) is then used by the system to update the means Operational Planning (070), for an optimal control of the continuation of the tests for the markers not yet tested.

At step d2), one propagates the value of relevance P (Gi) in the means of storage (051). The propagation consists, for each marker Gj of the set of the markers in (051) not yet tested or evaluated, in assigning to the relation between Gj and Gi the value of propagation:

PROPAGATION(Gi,Gj)=Relevance(Gi)(=P(Gi)),

which is stored in the means (051) in the entry corresponding to the relation between the markers Gi and Gj. Due to the fact that the relations are bidirectional (in other words the relation between Gi and Gj is the same one as between Gj and Gj), and as in this case Gj is not tested yet, by extension one can note indifferently PROPAGATION (Gi, Gj)=PROPAGATION (Gj, Gi)=P (Gi), which will be useful for the following.

At step d3), one re-initialized the Resource Unit, which causes to return it “FREE” and not configured, so that it can be re-used in a forthcoming cycle of stages (B, C, D, E).

FIG. 4 shows the sequence of operations carried out in the means (400).

FIG. 5 shows the contents of the means (051) of storage, at one given moment of the execution of the method, after the execution of the evaluation of the results of test of the marker Gi, the markers Gj, Gk and Gi being still not tested.

After the update of the means (051) of storage at the stage D), one updates the Operational Planning (070) at the stage E) of the method, as described further in present description.

### Stage E) for the Method

The stage E) aims at updating of the priority of the markers present in the means (051), allowing the system to manage in a dynamic way, and in real-time, the continuation of the execution of the method.

More precisely, calculations of relevance of the raw results initially generated by the Resource Units (061) are used by the system for, if necessary, modifying the set of priorities of the tests to be realized for the markers Gi not yet tested.

On a purely illustrative basis, if the result of a test for a given marker Gx is informative with respect to a situation of deregulation of the cellular division, the stage E), through propagation of the information, can modify test priorities for specific markers Gi not yet tested, said markers being informative on a situation of cancer, in a way that assigns an higher priority to these informative markers Gi, allowing the execution of the tests with these markers in priority.

The stage E) thus consists of an update, in real-time, of data contained in the system, in order to carry out the method in an optimal way, in particular in order to generate an informative set of state parameters for Gi markers with the shortest execution time.

At step e1) one carries out a new classification about the markers Gi not yet tested at this moment, and indexed in the means (051). The new classification is made by assigning to each marker Gi not yet tested a value of priority, the aforementioned value being the weight Pi obtained by an equation defined further. At step e2) one updates the data contained in Operational Planning (070) with the values of weight obtained at the stage e1) for each marker Gi not yet tested, the update being described thereafter.

More precisely, at any time during the execution of the method, the markers can be classified in two sub-sets, respectively (I) the sub-set of the markers already tested and (II) the sub-set of the markers who were not tested yet.

Preferentially, the system permanently maintains in the means (070) a classification of the markers in the second sub-set of markers above, i.e. the sub-set of the markers not yet tested. The establishment of this classification in a continuous way during the execution of the method allows a dynamic adaptation of the order in which the various unit tests for each Gi marker will be carried out, taking into account the results already generated for the sub-set of Gi markers already tested.

Preferentially, the adaptation in real-time of the order in which the various unit tests for each Gi marker not yet tested will be carried out is managed by the method of control (100), thanks to a specific means of storage for the order of the markers not yet tested, according to the priority assigned to them, the aforementioned means of storage consisting in the Operational Planning (070).

An illustration of the contents of the Operational Planning (070) is schematized on FIG. 6.

The Operational Planning (070) includes, for each marker Gi not yet tested at a given moment of the execution of the method, (I) a reference of identification of the said marker Gi, and (II) a value of priority for the said marker Gi.

The update of the contents of the Operational Planning (070) is carried out at the end of each execution of a test for a given marker Gi.

In other words, the stage E) of data update is carried out at the end of each execution in sequence of the stages b), c) and d) for a given marker Gi.

The calculation of weights is made by a numerical function WEIGHTS( ) positive or null which assigns to each marker Gi not yet tested, and contained in the means (051) a value WEIGHTS(Gi) calculated according to the following equation:

WEIGHTS(Gi)=Σ{METRIC(Gi,Gk)*PROPAGATION(Gi,Gk)}, (1<=k<=N),

in which:

N is the number of markers present in the means (051),

Σ is the operator “addition”

METRIC (Gi, Gk) is the metric value associated with the relation between Gi and Gk in the means (051),

PROPAGATION (Gi, Gk) is the propagation value assigned to the relation between Gi and Gk in the means (051) at the stage d2) during the test of the marker Gk, the value is null by default if Gk is not tested yet.

WEIGHT( ) is thus necessarily positive or null.

It is pointed out that the value of PROPAGATION is equal to zero at the beginning of the stage A) of initialization of the method, so that, for example, the relations between a marker Gj and any another marker Gi not yet tested or evaluated does not contribute to the weight of Gj.

After treatment of the totality of the sub-set of markers not yet tested or evaluated at the time of execution of E), the value of WEIGHT(GJ) determines the new priority of Gj in this sub-set.

At the stage E), the Operational Planning (070) is updated by the insertion of the calculated data of WEIGHT (Gi) of the markers not yet tested or evaluated. Thus, for any marker Gj not yet tested or evaluated, if the new value of WEIGHT(GJ) is greater than the current PRIORITY value of Gj in the (070), the PRIORITY value for Gj is fixed at WEIGHT(Gj) in the Operational Planning (070).

As one sees it on FIG. 2, when the method of control (100) receives a signal UPDATE, the means (100) carries out a calculation of weighting with the data contained in the means (051) of storage in order to carry out a new classification of the markers not yet tested or evaluated in the Operational Planning (070).

After the update of the Operational Planning (070), the next marker to be tested at the next execution of stage B), succeeding the stage E), is the marker having, in the means (070) the highest PRIORITY value. In certain cases, several markers referred in the means (070) will have the same value of PRIORITY. These markers will be treated by the method according to a random selection.

In certain modes of realization of the method according to the invention, the sub-set of markers not tested or evaluated yet at a given moment of the execution of the method, which are referred in the means (070), can be distinguished according to whether they belong to two smaller sub-set, respectively (I) a first sub-set of “SIGNIFICANT” markers having a PRIORITY value higher than a preset threshold of significance ACCEPT, and (II) a second sub-set on “NONSIGNIFICANT” markers having a PRIORITY value lower or equal to the preset threshold of significance ACCEPT.

Value ACCEPT is an arbitrary, positive or null value, which is defined by the user before an execution of the method, and which can in certain cases be adjusted dynamically by the user during the execution of the method. The value of acceptance ACCEPT fixes the relative sizes of the first and the second sub-sets of markers not tested or evaluated yet, which evolve during the execution of the method.

As that is described above, the content of the Operational Planning (070) is modified after each execution of a sequence of stages B) to D) of test, then evaluation, then analysis of the results for a marker Gi being tested. According to this characteristic of the method, the system is able to adapt dynamically the order of the tests which remain to be realized in an incremental way, according to the results already obtained with the markers Gi already tested and evaluated, during an execution of the method.

In certain specific modes of realization of the method, the user can initially and arbitrarily fix the PRIORITY values for a sub-set of markers Gi, before the stage A), this sub-set of markers being appointed “SEEDS” markers for purposes of this description. During the execution of the method, the initial values of PRIORITY fixed arbitrarily by the user for SEEDS markers will influence the beginning of the evaluation, in a transitory mode of operation of the method. Then, the system will auto-adapt as results of test are generated for these markers, with the progression of the execution of the method.

Preferentially, the values of PRIORITY which are given arbitrarily by the user to the SEEDS markers are positive, null or infinite positive, with the consequence that:
the SEEDS markers whose value of initial PRIORITY is infinite positive, are tested first in any case, at the beginning of the execution of the method, which constitutes a stable initial order;
the SEEDS markers for which the user fixed a strictly positive, but not infinite initial PRIORITY value arbitrarily are tested according to an decreasing order of value of PRIORITY. But the order of these SEEDS markers may be modified, according to the results obtained during the evaluation of the first SEEDS markers, as that was already described above.

In an ultimate way, when the user initially fixes a priority for each marker used in the method, i.e. all the markers are SEEDS markers, and when a null metric is specified (i.e. whatever the pairs of markers Gi and Gj, METRIC (Gi, Gj)=0), it results that the evaluation proceeds according to the order specified by the user.

It is pointed out that, at the beginning of the stage A) of initialization of the method, the markers Gi which are not SEEDS have a PRIORITY value zero.

Moreover, in the modes of realization of the method in which no value of arbitrary PRIORITY is affected for the totality of the Gi markers, then the value of PRIORITY is equal to zero for the totality of the Gi markers and the process begins its execution by a random selection of the first Gi markers to be tested.

The difference between the theoretical execution time of the stages B), C) and D), on the one hand, and the real execution time of these stages, on the other hand, grows with the ascending values of Nm. In particular, with high values of Nm, the analysis and execution time of the tests becomes a limiting factor.

In order to overcome this disadvantage, the method can be stopped prematurely, before the execution of tests for the totality of the markers Gi initially indexed in the means (051), either manually on the request of the user, or automatically when a condition of abortion occurs during the execution of the method.

In a general way, the method is stopped, manually or automatically, if the tests which were already carried out with a part only of the markers Gi are such as the combination of state parameters for markers already tested are sufficiently informative on the state of the set of cells tested.

On a purely illustrative basis, the method can be stopped in an automatic way in the case where, for a given marker Gi, the value of the state parameter generated by the Resource Unit (061) used to test the aforementioned marker Gi, is at least equal to, or most equal to, a value preset by the user. For example, if the method according to the invention is used to determine, among others, the inflammatory state of the set of cells tested, the method can be stopped automatically if the parameter of state of the marker “level of expression of gene coding TNFalpha” is at least equal to a given quantity of mRNA or cDNA coding TNFalpha.

### Self-Adaptation of the Method or System

As that was previously described in present invention, the Operational Planning (070) is a critical means used during an execution of the method according to the invention, since the means (070) contains, for each marker Gi selected at the stage A), a PRIORITY value for the aforementioned marker, that may dynamically evolve and influence the order in which each Gi marker is tested.

According to advantageous modes of realization of the method of the invention, a given execution of the method with an ensemble of markers Gi can benefit from results of tests carried out by one or more anterior executions of the method with markers that are present in the aforementioned set of markers Gi. According to these advantageous modes of realization of the method, the priorities of markers Gi to be tested during the execution of the method, as well as the METRIC values characterizing relations between pairs of markers Gi, are at least partially deduced from the results of tests carried out in an anterior execution of the method with concerned markers. In this case, the method or system according to the invention relies on a self-adaptation means, described below in an illustrative—but not limiting—mode of realization.

One considers an ensemble G of N markers Gi, identified G1, G2, . . . GN.

One considers also a set E of M former evaluations of each marker included/understood in set G of markers.

The self-adaptation means used by the method or the system according to the invention relies on the automated computation, given results of former evaluations, of a set of an arbitrary number P of seeds on the one hand, and of a metric on the other hand, identified MET_STAT in the following; the P seeds and the new metric MET_STAT being used during a new execution of the method of the invention to realize a (M+1)th evaluation of markers contained in the set G of N markers Gi aforementioned.

The self-adaptation means includes the following stages:

I) a stage during which, for each former evaluation Ei (with 1<=i<=M), a column vector Res(Ei) is generated, the aforementioned vector Res(Ei) containing N lines and only one column. In the aforementioned vector Res(Ei), Res(Ei)(j,1) (with 1<=j<=N) contains the result of evaluation (state parameter) obtained for the marker Gj of G during evaluation Ei, the aforementioned result being initially stored in the means (052) during the corresponding execution of the method for Ei. Alternatively, it is possible to form vectors Res(Ei) starting from data, for the markers of G, obtained in the specific literature or databases. Thus, in the example where the markers used are genes, it is possible to compose vector Res(Ei) starting from genomic data obtained in a database such as MIAME.
II) a stage during which a composite vector, indicated ResComp, is generated.

The ResComp is a column vector containing N lines and only one column. Line j (with 1<=j<=N) of the ResComp vector contains, for marker Gj present in G, the sum of the results (state parameters) generated by the M former executions of the method of the invention for the aforementioned Gj marker.

The value of ResComp is thus calculated according to the following formula:

ResComp=ΣRes(Ei), in which:
Σ represents the symbol of the vector sum;
Res(Ei) represents the vector column Res computed from fromer evaluation Ei for the set of markers G;
with 1<=i<=M.
   III) Using the composite vector ResComp above, one generates a matrix, called MATSTAT, which is a positive symmetrical matrix of N lines and N columns.

Matrix MATSTAT is defined by the following formula:

MATSTAT=ResComp*transposes(ResComp), in which:
the symbol “*” means the vector product operator, and
“transposes ( )” means the operator of matrix transposition.

MATSTAT is used to select, for the (M+1)th evaluation, an arbitrary number P of SEEDS markers (Gs1 . . . GsP) that are the P most relevant markers within the M former evaluations. These markers are the P markers Gs1 . . . GsP such as MATSTAT (Gs1, Gs1) . . . MATSTAT (GsP, GsP) are the P greatest values, by decreasing order, of the diagonal of matrix MATSTAT.

MATSTAT is then used to define the metric model MET_STAT for (M+1)th evaluation while choosing for any i (1<=i<=N), for any j (1<=j<=N) a metric value for the relation between Gi and Gj: MET_STAT(Gi,Gj)=MATSTAT(i,j). Owing to the fact that MATSTAT is symmetrical, it is checked that MET_STAT (Gi,Gj)=MET_STAT (Gj,Gi). One chooses then a model of relevance identical to that used for the M the preceding ones evaluations, then one carries out (M+1)th evaluation. The results obtained can then be integrated into the statistics the way described previously, to form the SEEDS and the metric model for the (M+2)th evaluation, and so on.

The self-adaptation is thus implemented in an incremental way, evaluation after evaluation of markers of G, during the successive execution of the method of the invention.

The scheme of self-adaptation described above was used in the accompanying example, with a set G of 51 genes used as markers, presented thereafter. In this example, one uses a collection of results already obtained for a series of distinct substances, in order to perform a statistical analysis of the results aiming at defining SEEDS and a metric model, as described previously in present description.

In the example with the 51 genes used as markers, one generated, during the statistical analysis, a symmetrical matrix MATSTAT of 51 lines and 51 columns. As described above, such a matrix MATSTAT is generated automatically during the execution of the method of the invention, without requiring intervention of the user.

On a purely illustrative basis, at the beginning of an execution of the method, user selects an arbitrary number Ns of markers “SEEDS”. The system selects then NS noted markers G1 . . . GNs such as the values corresponding to each one of these markers, on the diagonal of matrix MATSTAT, are the NS the to highest values of the diagonal in decreasing order:

MATSTAT(G1,G1)>=MATSTAT(G2,G2)>= . . . >=MATSTAT(GNs,GNs).

Thus, at this stage, the system successively assigns the values of priority to the SEEDS markers G1, . . . , GNs and stores the PRIORITY values in the means (070) accordingly, as that was already exposed previously in present description.

On a purely illustrative basis, the system stores in the means (070) the PRIORITY value 1000*MATSTAT (Gi, Gi) to the seeds markers Gi (with 1<=i<=Ns), and the value of PRIORITY of value 0 to all other markers.

Moreover, the system uses the values of metric present in matrix MATSTAT, by application of a metric model MET_STAT configured in (020):

Whatever (Gi, Gj) in all 51 marker,

MET_STAT(Gi,Gj)=MATSTAT(Gi,Gj)

At last, the model of relevance charged in (030) is the model PERT_STAT:

Whatever the result RES of a test carried out by any Resource Unit, specifically designed in the example to measure the level of expression for a gene G under precise conditions of exposure of a cell culture to a substance,

PERT_STAT(G)=0 if 0.5<RES<2;

PERT_STAT(G)=1 if not.

### Parallelization of the Tests

In certain modes of realization of the method, the means (100) of control can dynamically (I) add one or more Resource Units (061) in the set (060), (II) remove one or more Resource Units (061) from the set (060), or both.

In these modes of realization of the method, the same physical system can handle simultaneously several distinct executions of the method of evaluation according to the invention. For example, in these modes of realization, a single physical system can simultaneously carry out the determination of the effects of several distinct substances or compounds on a given set cells, or simultaneously carry out a determination of the effects of a given substance or a compound on several distinct set of cells, or even carry out simultaneously much more complex determinations, for example the effects of several substances, each one on a plurality of distinct set of cells, with different times of exposure.

In these modes of realization, each single process of evaluation, using the method according to the invention, and which is distinct from the other process of evaluation simultaneously carried out, uses means (051), (052) and (070) that are specific and private to the execution of this single process. Each process of evaluation also uses Resource Units (061) contained in a general set (060-G) of Resource Units (061) that is shared by all processes of evaluation simultaneously carried out. In these modes of realization, the general set (060-G) of Resource Units (061) can also be called “CLUSTER” (060-G) of Resource Units (061).

Each Resource Unit (061) is assigned of a single identifier, as shown on FIG. 7.

At one moment T, several sets (060) of Resource Units (061) are defined, each set (060) of Resource Units consisting of a sub-set of CLUSTER (060-G). At each moment a given Resource Unit (061) is present in at most one set (060) of Resource Units.

However, at one given moment T during the execution in parallel of the distinct processes of evaluation, a given Resource Unit (061) which was initially present in a set (060) of Resource Units assigned to carry out a process of evaluation P1 according to the invention can be removed from this set (060) dedicated to P1 and be added to a distinct set (060) assigned to carry out a process of evaluation P2 according to the invention.

Thus, in these modes of realization of the invention, the size of each set (060) of Resource Units (061) assigned to each processes of evaluation P1, P2, . . . , Pn can vary dynamically.

In these modes of realization, a means (1000) known as Monitor of Cluster controls and orders the assignment of each Resource Unit (061) of CLUSTER (060-G) to a single set (060) at a time of Resource Units selected to carry out a process of evaluation Pi according to the invention.

To this end, the Monitor of Cluster (1000) estimates, at every moment T, the number of Resource Units (061) which is necessary for the execution of a given process of evaluation Pi. The Monitor of Cluster applies the following general rule: the number of Resource Units (061) assigned at one given moment T to a set (060) for the execution of a process of evaluation Pi according to the invention depends on the number of markers Gi referred as being “SIGNIFICANT” with respect to the value of acceptance ACCEPT [defined at stage E) in the present description of the invention]. The number of SIGNIFICANT markers can be deduced, given ACCEPT value, from the state of the Operational Planning (070) used by Pn at this moment T.

Thus, if RP (T) is the number of Resource Units (061) necessary for the execution of the process of evaluation Pi at the moment T, and if SG (T) is the value of the number of “SIGNIFICANT” markers referred at this moment T in the Operational Planning (070) used by the aforementioned process of evaluation Pn, then:

RP(T)=O(SG(T)),

with:
Y=O(X) meaning that Y is linearly bound to X, in other words a linear function of X(Y=aX+b)

Thus, the more there exist “significant” markers in the Operational Planning (070) at a given moment T for a given process of evaluation Pi (1<=i<=n), the larger the number of necessary Resource Units (061) in the set (060) of Resource Units of Pi.

However, in an optimal parallel execution of a plurality of processes of evaluation P1, P2, . . . , Pn according to the invention, it is fair to bound the maximum value that RP(T) can take for any of the processes P1, P2, . . . , Pn in progress. This to prevent that the execution of given process of evaluation Pi, for which the Operational Planning (070) contains a very large number of “significant” markers, does not monopolize too many Resource Units (061) which would in turn not be available, although required, for the simultaneous execution of the other processes of evaluation P1, P2, . . . , Pn. The Monitor of Cluster (1000) thus implements a protocol of assignment of Resource Units (061) allowing an fair share of the Resource Units (061) between the processes of evaluation P1, P2, . . . , Pn.

The protocol of assignment of Resource Units (061) to the various sets (060) of Resource Units used by the simultaneous processes of evaluation P1, P2, . . . , Pn can be for example the protocol describes hereafter.

Given a CLUSTER (060-G) containing a number RU(T) (variable in time) of Resource Units, and N processes of evaluation Pi according to the invention [with 1<=i<=N] which are carried out simultaneously. A dynamic partition of the Resource Units (061) of CLUSTER (060-G) is carried out among the N sets (060) Rbi of Resource Units dedicated to the processes of evaluation Pi (1<=i<=N). To ensure a good progress of each process of evaluation, each set (060) of Resource Units (060) is affected of a minimal number of Resource Units. A fraction F (0<F<=1) of RU(T) is assigned to this minimum, so that each set (060) Rbi (1<=i<=N) of Resource Units (061) contains at least MINi(T)=F*RU (T)/N Resource Units (061) at the moment T.

The (1−F)*RU (T) remaining Resource Units (061) are distributed dynamically among the N sets (060) of Resource Units Rbi (1<=i<=N), according to the number of “significant” markers Sgi (T) referred at the moment T in the Operational Planning (070) used by each simultaneous process of evaluation Pi (1<=i<=N).

For each process of evaluation Pi, one allocates, in addition to the MINi(T) initial Resource Units (061), DYNi(T) additional Resource Units (061) at the moment T, with the following equation:

DYNi(T)=(1−F)*RU(T)*Sgi(T)/(ΣSgj(T), 1<=j<=N)

The arbitrary rule is introduced: if Sgi (T)=0, then Sgi (T)=1.

An alternative to the rule above is preferred in situations where each process of evaluation Pi uses a collection of markers Ci, such as the variance of Ci (1<=i<=N) is large, i.e. in situations where the number of markers referred in the means (051) and (052) of storage is highly variable from a process of evaluation Pi to another distinct process of evaluation Pj. In this specific situation, the minimum number of Resource Units (061) can advantageously be given by the following rule:

MINi(T)=F*RU(T)*(Ci/ΣCj, 1<=j<=N))

To each process of evaluation Pi, one allocates, in addition to the MINi(T) initial Resource Units (061), DYNi(T) additional Resource Units (061) at the moment T, with the following equation:

DYNi(T)=(1−F)*RU(T)*Sgi(T)*Ci/(ΣCj*SGj(T), 1<=j<=N)

The arbitrary rule is introduced: if Sgi (T)=0, then Sgi (T)=1.

The rules above are easily extended by the specialist to a number N(T) of processes of evaluation according to the invention which depends on time. The Fraction F can also depend on time, i.e. the Fraction F can be adjusted by the user at time T. At last, the number RU(T) of Resource Units (061) contained in CLUSTER (060-G) can vary in time in order to allow the user to dynamically adjust the size of CLUSTER (060-G) of Resource Units (061) by dynamic addition/removing of Resource Units.

Thus, another purpose of the invention consists of a method to simultaneously determine the state of a plurality of sets of prokaryotic or eukaryotic cells by means of the determination of the state of an ensemble of biological markers contained or expressed by the cells of each set of cells, the aforementioned method including the following stages:
1) to realize, for each set of cells of the plurality of sets of cells, a method according to the invention including the stages A) to F) described above;
2) to recover, for each set of cells, the set of the state parameters contained in the means (052) at the end of stage F) of the method carried out with the aforementioned set of cells, the set of the recovered data constituting the state of the plurality of sets of cells determined by the method;

As indicated previously in description, the expression “set of cells” includes the cell culture exposed in given environmental conditions. For example, a “set of cells” includes a cell culture of a given type, for example of a determined cellular line, placed under conditions of temperature, of gas atmosphere and given culture medium. Also, a “set of cells” includes cells of a given type which are exposed to a substance S, for example a candidate substance S to be tested. Also, a “set of cells” includes cells of a given type which are exposed to the aforementioned substance S during one given exposure time.

To carry out the aforementioned method, a plurality of executions of the general method according to the invention are carried out, each execution of the general method allowing to determine the state of an ensemble of cells, included in the plurality of sets of cells whose state is evaluated.

As that will be exposed further in present description, an execution of the general method according to the invention is carried out using a system to determine the state of at least one set of prokaryotic or eukaryotic cells, whose characteristics will be defined. To carry out the above method of determination of the state of a plurality of sets of cells in the shortest amount of time, it is advantageous to implement simultaneously a plurality of systems of determination of at least one set of cells, in order to simultaneously carry out as many executions of the general method of the invention than there are distinct sets of cells to be evaluated. In this particular mode of realization of the aforementioned method, one implements simultaneously, or nearly simultaneously, a plurality of systems according to the invention, the aforementioned plurality of systems according to the invention constituting a device according to the invention.

To carry out the aforementioned method of determination of the state of a plurality of sets of cells, the number of systems used will depend on the overall number of sets of cells to evaluate as well as economic considerations.

Indeed, in certain modes of realization, it can be advantageous to carry out a number of systems according to the invention which is lower than the number of sets of cells to test, for reasons of costs of each system. In these modes of realization, the process above will be initiated with a limited number of systems according to the invention, in order to carry out as many executions of the general method and to determine the state of the said number of sets of cells, lower than the number of the totality of the sets of cells to evaluate. Then, after the ending of each first execution of the general method on a given system, an following execution of the method is started, in order to evaluate the state of the set of cells not tested yet. In these particular modes of realization, a device according to the invention, allowing the determination of the state of the totality of the sets of cells to test, includes a number of systems according to the invention lower than the number of sets of cells to be tested.

In certain modes of realization of the method according to the invention, test execution priority data contained the Operational Planning (070) is initialized in a way that induces a total order of markers Gi, the method using null metric so that the chronology of the markers tested during the execution of the method accurately reproduces the specified initial order.

In some other modes of realization of the method according to the invention, execution priority data contained the Operational Planning (070) for whole or part of the markers contained in (051) are initialized with positive or infinite positive values of priority, the method using an arbitrary metric and an arbitrary model of relevance.

The method according to the invention can in particular be applied to the determination of the effect of a substance S to test on an ensemble CELL of prokaryotic or eukaryotic cells. In this particular mode of realization of the method, the effect of the substance S on the set of cells CELL can be tested (I) for several values of concentration (C1, C2, . . . , Cn) of the substance S for one exposure time T, (II) for several values of exposure time (T1, T2, . . . , Tn) of the set of cells CELL to the substance S for a value of concentration C of S or (III) for a combination of concentration C and exposure time T. In this particular mode of realization of the method, the stages A) to E) are carried out for a value of concentration C of the substance S and a value of exposure time T of the cells CELL to the aforementioned substance S, during an execution of the method. Thus, each execution of the method allows the determination of the state of the set CELL of prokaryotic or eukaryotic cells, for a given combination of concentration C of S and exposure time T of the cells CELL with S. Thus, to determine the effect of the substance S on the set of cells CELL, one carries out as many executions of the method than there exist combinations of values of concentration C with the values of exposure time T. The number of useful combinations is in general fixed by the user, before the starting of the evaluation process. The number of combinations is fixed in particular according to the volume of tests results required to finally obtain of a statistically significant knowledge of the effect of the substance S on the set of cells CELL. The specialist of the profession can easily fix the number of combinations required to obtain a statistically significant result, by having recourse to his technical general knowledge.

The plurality of executions of the method which are necessary to draw out a complete determination of the effect of the substance S on the set CELL of prokaryotic or eukaryotic cells can be realized (I) simultaneously, (II) in sequence or successively in time or even (III) simultaneously for part of the number of executions of the method and successively in time for the remaining part of the number of executions of the method.

Thus, the present invention also has as an aim a method to test the effect of a substance S on an ensemble CELL of prokaryotic or eukaryotic cells including the following stages:
a) to determine a number of combinations of (I) concentration C of the aforesaid the substance S and (II) of exposure time T of the set of cells CELL to the aforementioned substance S to carry out the test;
b) to carry out as many executions of the general method of the invention than there are combinations determined at stage a);
c) to determine the effect of the substance S on the set CELL of prokaryotic or eukaryotic cells, the aforementioned effect being determined from totality of the state parameters of the markers Gi contained in the means of storage (052) after completion, at the stage b), of the last execution of the general method of the invention.

Thus, the invention also has as an aim a method such as the one defined above, having for finality the evaluation of the impact of a substance S disturbing a cell culture CELL under conditions of concentration and duration (Cref, Tref), and taking into account the results of a set of N former evaluations {E1 . . . IN}, knowing that:
each evaluation Ei (1≦i≦N) was obtained by a system Sys(i) according to claims 10 and 11
each system Sys(i) (1≦i≦N) aimed at the evaluation of the impact of a substance Sub(i) identical or not to S, disturbing a cell culture C(i) identical or not to C under conditions of concentrations and duration (Cs(i), Ts(i)) identical or not to (Cref, Tref) by means of a whole of marker Gset(i),
   so that the method can define by third party means, for a sub-set M of markers Gi of UNION (Gset(i)), (1≦i≦N), UNION representing the set union operator, a weighting POND_M of the markers Gi of M, the weight of each marker G of M in POND_M taking into account the values RELEVANCE(G), Result(G) and Iter(G) obtained from the means (051) and (052) used in evaluations Ei (1≦i≦N) in which G took part, the weight of each marker G of M in POND_M representing the relevance, globally taken, of the marker G in the N evaluations {E1 . . . EN}.

The invention also has as an aim a method as described above using, in order to carry out the evaluation of the impact of the substance S on the cell culture CELL under the conditions of concentration and exposure (Cref, Tref), a set Gset of biological markers containing a sub-set M of markers known to have taken part in N former evaluations Ei (1≦i≦N), where the weighting POND_M of whole or part of the markers of M can be established by the system according to description given in claim 12, weighting information allowing the configuration of initial priorities data for the markers of M in the Operational Planning (070) of the said system, the aforementioned initial priority of each marker Gi of M being proportional to the weight of the marker Gi in weighting POND_M.

The invention also has as an aim a method as described above, using, in order to carry out the evaluation of the impact of the substance S on the cell culture CELL under the conditions of concentration and exposure (Cref, Tref), a set Gset of biological markers containing a sub-set M of markers known to have taken part in N former evaluations Ei (1≦i≦N), where the weighting POND_M of whole or part of the markers of M can be established by the system according to the description given previously, weighting information allowing the configuration of metric relations between markers of M in the Metric Model (020) of the said system, the metric value of the relation between two unspecified Gi markers and Gj of M being obtained by comparison of the weights of the markers Gi and Gj obtained in POND_M.

The invention also has as an aim a method as defined above using an ensemble of markers Gi, the method being used to carry out multiple evaluations of impacts of arbitrary substances on arbitrary cell cultures under arbitrary conditions of concentration and exposure time with whole or part of the markers Gi, the method accumulating and correcting weighting data of Gi markers during successive evaluations, thus allowing the self-adaptation of initial priorities data and metric data for whole or part of the set of markers Gi used.

Thus, the general method of determination of the state of at least an ensemble of prokaryotic or eukaryotic cells according to the invention can be also applied to the determination of the effect of a substance S on a plurality of cell types (CELL1, CELL2, . . . , CELLn), for example to a plurality of cultures of cellular lines and/or cultures of cells in primary culture. To carry out such a test, one determines as a preliminary (I) the number and the identity of each set (CELL1, CELL2, . . . , CELLn) of cells to be tested, (II) the number of values of concentration (C1, C2, . . . , Cn) of the aforesaid the substance (S) to test and (III) the number of values of exposure time (T1, T2, . . . Tn) of the cells to the aforementioned substance S, then one determines the number of combinations of these three parameters which are necessary to carry out the aforementioned test. Then, one carries out as many executions of the general method of determination of the state of at least one set of cells according to the invention than there are combinations determined above for the three parameters of the type of cells, concentration and exposure time. The number of combinations required to obtain a statistically significant result can be easily given by the specialist of the profession, using his technical general knowledge.

The invention thus has also as an aim a method to test the effect of a substance S on a plurality of sets of prokaryotic or eukaryotic cells including the following steps:
a) to determine a number of combinations of (I) sets of cells (CELL1, CELL2, . . . , CELLn) contained in the aforementioned plurality of whole of cells, (II) values of concentration (C1, C2, . . . , Cn) of the aforesaid the substance S and (III) of exposure time (T1, T2, . . . , Tn) of the cells to the aforementioned substance S;
b) to carry out as many execution of the general method of the invention than there are combinations determined at the step a);
c) to determine the effect of the substance S on the plurality of sets of prokaryotic or eukaryotic cells, the aforementioned effect being deduced from totality of the state parameters for the markers Gi contained in the means of storage (052) after completion, at the step b), of the last execution of the general method according to the invention.
   Illustrative Examples of Execution of the Method with Self-Adaptation and Parallelization of the Tests

The characteristics of the method according to the invention allowing (I) a self-adaptation of the system with a growing number of executions of the method and (II) a possibility of simultaneously carrying out distinct executions of the method, were described previously.

Some modes of realization of the method are presented below, which illustrate technical advantages related to the self-adaptation and the possibility of parallelization of the tests, in particular when the method according to the invention is applied to the determination of the effect of a substance or a compound to be tested on a prokaryotic or eukaryotic cells set.

Thus, in some modes of realization of the method according to the invention, the operator can optimize the execution duration of the method, for example by informing the system about (I) the type of activity sought for the compound to be tested or about (II) the type of chemical structure of the compound to be tested, before, or simultaneously at, the initialisation step A) of the system preceding the start of the test.

In some modes of realization of the method, the introduction of one or more data relative to the compound to be tested, in particular the type of activity sought for the aforementioned compound or its type of chemical structure, makes it possible to select among former executions of the method those which aimed to test compounds sharing some common characteristics (structure and/or activity) with the compound to test. The use of the results of these former instances of execution then makes it possible to build a MATSTAT matrix used for the loading of the means (070) of Operational Planning and the means (020) of storage of the metric values of the relations between markers, at the step a1) as already detailed in the description of the capability of self-adaptation of the invention.

More precisely, when one wishes, for example, to test the cytotoxicity of a given compound, one can use, at the step a1), the data stored in a MATSTAT matrix built from results of former instances of execution concerning markers relevant to cytotoxicity tests in order to define the values of metrics contained in the storage means (020) and the initial priorities of the seed markers contained in the means (070). One recalls that, in this case, the data contained in the used MATSTAT matrix were calculated with results of former instances of execution of cytotoxicity tests, (I) either with compounds whose cytotoxicity was already known at the time of the aforesaid former executions, (II) or with compounds which were found as cytotoxic during the course of former executions, (III) or with compounds of types (I) and (II).

An illustration of such a mode of realization of the method according to the invention is presented in example 2. In this example, the method of the invention was used to test the cytotoxicity of compound Paraquat, while using, to load the means of Operational Planning (070) with an ensemble of potentially suitable markers priorities data and the means (020) with the metric values potentially suitable for the relations between markers, results obtained from former executions of the method with 14 compounds known or determined as being cytotoxic, the aforementioned results being gathered in a MATSTAT matrix, under a format exploitable by the method.

In these modes of realization of the method of the invention, one can also load the means of Operational Planning (070) and the means of storage (020) at the step A), with a series of markers chosen on the basis of chemical structure of the compound to be tested. In these specific modes of realization of the method, the step of initiation of the system A) can (I) include a step of analysis of structure/activity relationships based on the structure data of the compound to be tested or (II) to be preceded by such a step of analysis of structure/activity relationships. In these modes of realization, the step of analysis of structure-activity relationships between (I) on the one hand, structure data of the compound to be tested and (II) on the other hand, structure data of compounds already tested by the method:
(1) allows to select one or more compounds already tested, providing a basis for relevant comparison with the compound to test, then to built a MATSTAT matrix with the results obtained for these compounds by the previous executions of the method, and
(2) allows to determine the identity and initial values relative to the Gi markers, starting from MATSTAT matrix previously generated. The data of the said selected markers Gi are then loaded in the means (070) of Operational Planning and the storage means (020) for the metric values of the relations between markers, at the step a1) of the method according to the invention, as already detailed in the description of the self-adaptation property of the invention.

Thus, according to some aspects of the method of the invention, the stage A) includes a step a) (iii-1), which preferably precedes the step a) (iii), the step a) (iii-1) consisting in a step of priority attribution for at least part of the Nm markers Gi for which the data are stored in the means (070) and (020), the aforementioned step a) (iii-1) being selected among one of the following steps:
(1) a step of a priority attribution to each marker Gi, this priority being calculated according to the degree of relevance of the Gi marker with respect to the kind of cellular disturbance searched during the current execution of the method;
(2) a step of attribution of a priority rank for each Gi marker, this priority rank being calculated according to the degree of relevance of the Gi marker with respect to the chemical structure of the compound S tested during the current execution of the method.

As already indicated, the relevance of each marker Gi (i) with respect to a type of cellular disturbance or (ii) with respect to the chemical structure of the compound tested, for the attribution of a priority rank to marker Gi, is calculated starting from the data already contained in MATSTAT matrix generated after former executions of the method, for example:
(I) former executions of the method aiming at testing a given type of effect of cellular disturbance, such as the oncogenicity, the inflammatory activity, the capacity to induce a cellular apoptose, the cytotoxicity, etc; or
(II) former executions of the method aiming at testing a compound having a given type of chemical structure, similar to the chemical structure of the compound tested in the current execution of the method. The similarities of chemical structure between two compounds, respectively (a) a compound for which test data are included in a MATSTAT matrix previously generated and (b) the compound to be tested, are preferentially calculated according to an analysis of the type QSAR.

The degree of relevance of a given marker Gi with respect to the test to be realized, for the attribution of a priority rank for this Gi marker, can be calculated by any means, as soon as this calculation makes it possible to allot a priority rank for this Gi marker. For example, for the attribution of a priority rank on the basis of chemical comparison of structures, the degree of relevance is provided by the result of the calculation of QSAR analysis.

Thus, in some modes of realization of the method, the step a) (iii-1) above consists of a step of attribution of a priority rank for each marker Gi, this priority rank being calculated starting from the results of an QSAR analysis between the chemical structure of the tested compound S and the chemical structure of compounds tested during former instances of execution of the method.

Typically, an QSAR analysis includes the following steps:
(1) to generate and store in a adapted storage means, in the memory of a computer, a set of data characteristic of the structure of a compound (S1) to be tested;
(2) to compare this set of data generated at step (1) above with a plurality of sets of characteristic data of the structures of a plurality of compounds (S2, S3, . . . , Sn), each set of data being characteristic of the structure of a given compound, among S2, S3, . . . , Sn, the aforementioned plurality of data sets having been generated before;
(3) to classify the compounds (S2, S3, . . . , Sn) according to the degree of similarity of structure of each one of the aforesaid compounds with the compound (S1) tested;
(4) to select the compounds having the greatest degree of similarity of structure with the S1 compound tested;
(5) to determine, for the compounds selected at step (4), the markers Gi whose presence and/or level of expression were modified, and to select the aforementioned markers Gi;
(6) to use the aforementioned markers Gi selected at the step (6) to initialize the system, at the stage A) of the method.

The ensemble of data of structure/activity relationship for compounds (S2, S3, . . . , Sn) above can result of:
results of tests obtained during the realization of former executions of the method of the invention with each one of the aforesaid compounds S2, S3, . . . , Sn; or
data of structure/activity relationship already known in the literature for each one of the aforesaid compounds S2, S3, . . . , Sn.

In accordance with the invention, the set of data above for each compound S2, S3, . . . ,Sn can be included in matrices of the type MATSTAT.

As already mentioned, information about marker priorities, generated for at least part of the Nm markers Gi, are loaded in the means (070) of Operational Planning.

An illustration of such a mode of realization of the method of the invention is presented in example 3. Example 3 describes the results of the implementation of the method for a test of evaluation of the cytotoxicity of a compound to test, Rotenone. A preliminary analysis of structure-activity relationship by QSAR (for “Quantitative Relationship Structure-Activity”) made it possible to determine that Rotenone was statistically close to several compounds, respectively Abamectine, Carbaryl and Fenazaquine. Abamectine, Carbaryl and Fenazaquine consist of reference compounds which had been tested already for their neurotoxicity with the method of the invention. The results obtained for Abamectine, Carbaryl and Fenazaquine make it possible to calculate a corresponding matrix MATSTAT. On the basis of data of identity, of priority rank of the Gi markers, and of metric of the relationships between markers contained in matrices MATSTAT generated for the neurotoxicity of Abamectine, Carbaryl and Fenazaquine, one selects the potentially relevant Gi markers for Rotenone to be tested, then one loads Operational Planning (070) and the Metric Model (020), at the stage a1) of the method according to the invention, as already detailed in the description of the property of self-adaptation of the invention.

The results obtained show that, thanks to the preliminary selection of the relevant Gi markers, a complete or almost complete information concerning the neurotoxic activity of Rotenone is obtained after only 23 cycles of execution of the stage B) to E) of the method (87% of the relevant informative markers tested) or after only 40 cycles of execution of the stages B) to E) of the method (100% of the relevant informative markers tested).

Thus, the realization of the step a1) of the method with a selection of markers for which initial priorities and the metric of the relationships between markers is determined on the basis of data contained in matrices MATSTAT previously generated (i) for compounds having the physiological effect that one seeks to determine for the candidate compound, and (II) for compounds having a structure close to the candidate compound, in particular those having also the aforementioned physiological effect (analyzes of structure-function relationships), makes it possible to reduce considerably the number of cycles of execution of the steps B) to E) of the method, which are necessary to generate the results allowing the classification of the candidate compound, for example among the neurotoxic compounds or the not-neurotoxic compounds.

Of course, during the execution of the method with the new candidate compound, a new corresponding matrix MATSTAT is generated, whose data could be later used as references for the selection of relevant markers Gi, at the stage a1) of a later execution of the method. Thus, the performances of the method of the invention increase with the growing number of executions already carried out, which made it possible to generate matrices MATSTAT of reference.

Globally, the characteristics of self-adaptation of the method according to the invention, for which certain modes of realization are illustrated above, induce an important saving of time, while reducing the number of execution cycles of stages B) to E) required to obtain a meaningful information, which also reduces the cost of implementation.

Also, according to certain modes of realization of the method according to the invention, to carry out the test of the physiological effect of a compound candidate, several distinct batches of relevant markers Gi may be used, for example a batch of relevant markers Gi for a cytotoxic effect by apoptosis, or a batch of relevant markers Gi for a cytotoxic effect by oxydative stress. As an illustration, for the test of a compound having possibly a physiological effect, several distinct selections of batches of relevant markers Gi may be used, for example a batch of relevant Gi markers for hepatotoxicity, or a batch of relevant Gi markers for neurotoxicity.

Consequently, for a compound to be tested for a given physiological effect, for example of cytotoxicity, several executions of the method according to the invention can be implemented, successively, i.e. in series, or simultaneously, i.e. in parallel.

According to another aspect of the invention, test results for a candidate compound, registered and analyzed successively in each cycle of the stages B) to E) in a given execution of the method, can allow:
(I) to determine if termination step F) can occur, before tests of all initially selected markers Gi are completed; and
(II) to decide, for the aforementioned candidate compound, to carry out one or several distinct executions of the method, with distinct selections of markers Gi, if necessary with one or more distinct selections of markers that are more relevant than the selection of markers Gi used in the first execution of the method.

This other aspect of the invention is pertaining to situations in which the results of tests obtained using the initial selection of markers Gi have low informative value and thus one needs to carry out further instances of execution of the method, with different and potentially more relevant selections of markers Gi. This type of situation is classically met when an instance of execution of the method is started whilst few informative MATSTAT matrices, or no MATSTAT matrix at all, relevant for the test to be realized, are stored in the system. This is typically the case when, for the test of a given physiological effect of a candidate compound, no reference MATSTAT matrix pre-exists and where the markers Gi are selected in a purely arbitrary way, or are selected on the basis of a priority rank derived from existing public knowledge of each marker Gi, which were not experimentally tested by the method of the invention.

According to this same aspect, supplementary executions of the method, used to further test the effect of the candidate substance, can be started and carried out simultaneously, successively, or delayed in time. The possibility of carrying out in parallel a plurality of instances of execution of the method of the invention was described previously in present description.

Still according to this particular aspect of implementation of the method according to the invention, the results of the tests carried out during a given execution can provide sufficient information to characterize the candidate compound, for example concerning his possible cytotoxicity properties. Also, the information necessary to characterize the candidate compound can be derived from the results of several instances of execution, among the plurality of executions of the method which are finally carried out with the candidate compound. In other cases, information required to characterize the candidate compound is derived from results obtained from all executions of the method finally carried out with this compound. As indicated previously, each execution of the method can be realized for the totality of the markers Gi initially selected for this instance of execution, or for only part of the markers Gi initially selected, in the event of early termination of this execution.

Lastly, still according to this same aspect of the method of the invention, in case a plurality of executions of the method is carried out for the determination of a given physiological effect of a candidate compound to be tested, the data of a MATSTAT matrix generated during the execution of a given instance of the method is taken as a matrix of reference, on the basis of which a distinct selection of markers Gi can be made to start another execution included in the plurality of executions mentioned above.

Thus, an execution E1 generates results which can then be used to select the markers Gi to be tested in an execution E2 of a plurality E of executions of the method. In certain case, it is the set of results generated by the realization of the plurality E of executions (E1, E2, . . . , En) which allows to characterize the candidate compound, from the point of view of the physiological effects to be determined.

One thus understands that, according to this particular aspect of the method, the nature and the sequence of the various executions of the method following the first of instance of execution (E1) are not necessarily known in advance, at the time of the starting the instance of execution E1. The nature and the sequence of the executions following the first instance (E1) can be set, partly or entirely, on the basis of result of tests generated during the execution of the first instance E1.

In certain cases, the successive determination of the collection of markers Gi used in each individual execution of the plurality of executions of the method, in particular by taking into consideration results generated by executions already carried out and included in the final plurality of executions, can be represented in the form of a tree of results. The said tree of results can itself be indexed in an additional means of information storage of the system of the invention. The data constitutive of this tree of results, stored in a suitable storage means of the system according to the invention, can be used later on, in a further execution of the method, and contribute for example in selections of sets of markers Gi for each execution in a plurality of executions of the method, for example to test another compound candidate, for an identical physiological effect.

Moreover, a tree of results generated during successive selections of sets of markers Gi in each execution participating in the plurality of executions of the method, (for example if each execution is carried out, for a given candidate compound, for a combination of parameters (i) of markers Gi to be tested, (ii) of concentration C of the compound, (iii) of the exposure duration T of the cells to the compound and (iv) of the identity of tested cells), can be represented in a vectorial assembly form. The parameters (trajectories) of the vectors constitutive of the tree of results can be defined according to one or more of the following vectorial representations:
(i) the vector of the results of all markers Gi for a given concentration C of the candidate substance and a given exposure time T of the cells to the substance candidate (trajectory in the space of the markers);
(ii) the vector of the results of a marker Gi for a given concentration (C) of the substance candidate, for all the exposure times (T1, T2, . . . , Tn) of the cells to the substance candidate (trajectory in the space of the concentrations);
(iii) the vector of the results of a marker Gi for all the concentrations (C1, C2, . . . , Cn) of the substance candidate, for a given exposure time (T) of the cells to the substance candidate (trajectory in the space of exposure durations);
(iv) the vector of the results of a marker Gi for all the concentrations (C1, C2, . . . , Cn) of the substance candidate and all the exposure times (T1, T2, . . . , Tn) of the cells to the substance candidate, for a given type of cell (CELL), for example for a given cell line (trajectory in the space of the cell lines)

Thus, in certain modes of realization of the method, one can generate a plurality of vectors (a plurality of trajectories) for the evaluation of single substance candidate.

These vectors (trajectories) can be compared to each other in order to determine various parameters of comparison, in particular among the parameter of comparison illustrated below:
(i) determination of the global evolution of the totality of the markers Gi tested (comparison between the trajectories the space of the markers);
(ii) determination of the evolution of a marker Gi for a plurality of concentrations of the candidate substance, in relation to the exposure time of the cells to that candidate substance (comparison of the trajectories in the space of the concentrations, for a given marker Gi);
(iii) determination of the evolution of each marker Gi in relation to the concentrations of the candidate substance, for a given exposure time of the cells to that candidate substance (analysis of the synchronization of the cellular response in relation to the concentration of the candidate substance, by comparison between the trajectories in the space of the exposure durations, for different markers Gi);
(iv) determination of the evolution of each marker Gi in relation to the exposure time of the cells to that candidate substance, for a given concentration of the candidate substance (analyze of synchronization of the cellular response in relation to the exposure times of the cells to that candidate substance, by comparison between the trajectories in the space of concentrations, for distinct Gi markers);

### Deregulation Space

The results obtained thanks to the implementation of a plurality of executions of the general method according to the invention allow the generation three dimensional matrices. Their data delimit a “space of deregulation”.

For example, a plurality of executions of the general method according to the invention allow to generate, for each marker Gi tested, concerning the effect of a candidate substance S on the level of expression of that marker Gi, data parameterized by, respectively:
(i) concentration value of the candidate substance S on all cells tested (a first dimension);
(ii) exposure time of all cells tested to the candidate substance S (a second dimension); and
(iii) the overall type of cells exposed to the candidate substance S (a third dimension).

This “deregulation space” delimits a space from which one can determine, in particular, the following information, valid for the marker Gi under consideration:
the space within which the said substance S induces an effect on the level of expression of the marker; Gi
the space within which the effect induced by S is reversible, this space being a sub-space of the space considered above;
the space within which the effect induced by S is irreversible, this space being also a subspace of the space considered above.

The knowledge, for each marker Gi, of the space of deregulation corresponding for a given substance S provides an additional tool, thanks to the method of the invention, allowing to summarize the relevant information on the activity of substance S (cytotoxicity, pharmacological activity, etc), which can be of a great utility, in particular for the purpose of legal homologation of substance S.

### System According to the Invention

The present invention also has as an aim a system to determine the state of at least an ensemble of prokaryotic or eukaryotic cells by means of the determination of the state of an ensemble of biological markers contained or expressed by the aforementioned cells, the aforementioned system including/understanding:
1) a data storage means known as Marker Base (010) including a set of data characterizing a number Nm of biological markers Gi;
2) a data storage means known as Metric Model (020) characterizing any relation Rm between two biological markers Gi among the Nm biological markers indexed in the means (010), the aforementioned means (020) containing for each relation between two biological markers present in (010):
   a reference of identification of a first biological marker G1 present in the means (010);
   a reference of identification of a second biological marker G2 present in the means (010);
   a numerical value METRIC(Rm) defining the metric of relation Rm between the first and the second marker;
3) a means (060-G) containing a number Nru of Resource Units (061), where each Resource Unit (061) is able to carry out and transmit at least one state parameter of a biological marker Gi, contained or expressed by a cell culture C, towards an evaluation means (300), each Resource Unit (061) including:
   an identification means (0611) of the aforesaid Resource Unit (061);
   a status means (0612) that indicates at any time the operating state of the aforesaid Resource Unit (061);
   a means (0613) of determination of at least one state parameter of a biological marker Gi, contained or expressed by a cell culture C;
   means (0614) of signal transmission between the aforementioned Resource Unit (061) and the evaluation means (300);
4) a data storage means known as Results and Relations Database (050) containing information about of biological markers Gi, contained or expressed by a cell culture C, the aforementioned means (050) including:
   a data storage means known as Marker Relation Database (051) containing information about relations between at least several biological markers selected in the group of the Nm biological markers indexed in the means (010), the aforementioned information consisting of, for each biological marker indexed in (010):
   a reference of identification of a first biological marker Gi present in the means (010);
   a reference of identification of a second biological marker present in the means (010);
   a value METRIC (Rm) defining the metric of the relation between the first and the second marker; and
   a value P(Gi) defining the relevance of the first marker in a test carried out for the given marker Gi and an ensemble of cell culture determined, the aforementioned value P(Gi) being calculated by an analysis means (400);
a data storage means known as Marker Results Database (052) of containing results of tests carried out by one or more Resource Units (061) contained in the Resource Unit Bank (060), the aforementioned Marker Results Database (052) containing a set of test results, each test result containing:
   an identifier of a biological marker present in the means (010) that has already been tested by a Resource Unit (061) contained in (060), and for which at least one state parameter value has been measured;
   a ranking value specifying the position of test of the said biological marker in the evaluation in progress;
   at least one state parameter value defining the result of the test carried out with the aforementioned biological marker by the aforementioned Resource Unit (061).
5) an evaluation means (300) used to evaluate results of tests carried out by one or more Resource Units (061) included in the means (060), the aforementioned means (300) comprising:
   data storage means for at least one state parameter coupled to a biological marker Gi, the aforementioned state parameter being generated by a Resource Unit (061);
   means to receive and transmit a signal between the aforementioned means (300) and Resource Units (061) present in the means (060);
   means for ordering the execution of tests by one or more Resource Units (061);
   means to transmit a signal from the scheduler (100) towards the aforementioned means (300);
   means to transmit a signal from aforementioned means (300) towards a means (400) of analysis of the results of test.
6) a means known as Relevance Model (030) for the storage of a function of calculation of a value P(Gi) defining the relevance of a biological marker Gi indexed in the means (010);
7) an analysis means (400) for the calculation of the value P (Gi) defining the relevance of a biological marker Gi indexed in the means (010) for a given cell culture C; the computation using the function stored in the means (030) with at least one state parameter defining the result of the test carried out with the aforementioned biological marker Gi by a Resource Unit (061), the aforementioned state parameter being provided to the means (400) by the means (300), the aforementioned means (400) containing a means of reception of a signal transmitted by the means (300);
8) a means known as Operational Planning (070) including:
   a set of test execution priority data for the biological markers Gi, the aforementioned means (070) containing, at every time of the evaluation and for each biological marker (Gi) not yet tested:
      an identifier of the said biological marker Gi for identification in means (010); and
      a value PRIORITY defining the level of priority of the said biological marker Gi;
   means of transmission of signals between the aforementioned means (070) and the scheduler (100);
   means allowing the initial configuration of the priority data for each marker used; data of priority being numerical positive or null values
9) an Initialization means (200) of the system, including:
   transmission means of a signal between the aforementioned means (200) and the method of control (100);
   transmission means of a signal between the aforementioned means (200) and the means (010) containing the set of data characterizing a number Nm of biological markers Gi;
   transmission means of a signal between the aforementioned means (200) and the Metric Model (020) characterizing a metric relation RM between two biological markers Gi
   transmission means of a signal between the aforementioned means (200) and the Results and Relations Database (050) containing state parameters of biological markers contained or expressed by a cell culture C.
   transmission means of a signal between the aforementioned means (200) and the Operational Planning (070) containing a set of data characterizing the initial priorities of the Nm biological markers.
10) a method of control, known as Scheduler (100) including:
   transmission means of a signal between the aforementioned means (100) and each Resource Unit (061);
   transmission means of a signal between the aforementioned means (100) and the Operational Planning (070);
   transmission means of a signal between the aforementioned means (100) and the means (300) of analysis of the test results;
   transmission means of a signal between the aforementioned means (100) and the Results and Relations Database (050);
   means of controlling operations of the Resource Units (061).

Advantageously, the system of the invention is carried out using a system including one or more computer(s). Thus, advantageously, the system such as defined above includes at least a computer, which includes the following internal and external components.

The internal components of the said computer system include a processor element, for example a microprocessor, which is inter-connected with a memory element. For example, the computer system can consist of a processor of the Pentium® type, such as a Pentium® microprocessor marketed by the Intel Company (U.S.A.), set at 3.20 GHz, the said microprocessor being connected to a main memory of a size of 256 MB or more.

The external components include a means of mass storage, such as one or more hard drives, with a storage capacity of at least 40 GB. The external components include also at least one peripheral, such as a printer or a computer screen. The external components include also at least one means of communication with the system, such as a computer keyboard, a mouse, graphic pad, etc

The external components include also at least one interface allowing the signals transmitted by the Resource Units (061) to be interpreted and processed by the microprocessor. Such an interface consists in general of a device able to convert an analogical signal generated by the Resource Units (061) into a numeric signal which can be processed by the microprocessor.

The external components can be remotely controlled by the computer by means of any suitable communication mechanism including, but unrestricted to: data-processing network, data busses, field busses, serial links etc. . . . In the case several computers are used, those can communicate through any means of communication available, including, but unrestricted to: one or more local network(s), series or parallel interfaces, wide area (wire or hertzian) network (Internet), telecom network, in order to allow a distributed and co-operative realization of the system.

In the system above, the means (010), (020), (030), and (050), consist of means of data storage which are preferentially included in the main memory of the computer system, more precisely in partitions of the main memory which are allocated by the microprocessor to these means of data storage.

In the system above, the method (100) of control, the means (200) of initialization, the means (300) of evaluation of the tests, the means (400) of data analysis and the means (070) of Operational Planning include also means of data storage which are preferentially included in the main memory of the system of computer. The data processing which is carried out by the means (300), (400) and (070), in particular the commands and computations, are preferentially carried out by the microprocessor.

When several computers are used, the means (010), (020), (030) as well as the means of storages used by the means (100), (200), (300), (400) and (070) can be distributed in the random access memories and/or in the storage memories of the said computers. In this case again, the processing of data and of instructions included in the ensemble of the means presented in the invention can be carried out on only one computer, or on the contrary be distributed on the ensemble of computers by means of distributed algorithms.

The computer system also includes, loaded in its main memory or in the means of mass storage, one or more elements of computer programs. The element(s) of computer programs include the software which is responsible for the management of the system according to the invention, in particular for the coordination of the operation of the internal and external components of the said system, and for the operating system containing the specific instructions for the implementation of the method according to the invention. Thus, the elements of computer program include series of instructions which allow the microprocessor to carry out the necessary data processing for the execution of the method according to the invention.

Moreover, in order to facilitate the implementation and the utilization of the method, the system can rely on additional software tools such as, but unrestricted to, databases software, interactive Human Machine Interfaces, archiving systems, in order to implement the ensemble of means presented in the invention.

In certain modes of realization of a system according to the invention, which are particularly adapted to the implementation of the method including a stage a) (iii-1) of attribution of priorities for the markers Gi, based on a comparison of the chemical structure of a candidate compound to be tested with the chemical structure of already tested compounds for which test results are included in already generated matrices MATSTAT, the said system includes moreover a means of comparison of chemical structures. For example, the said means can consist of a computer program including a series of instructions for realizing a comparative analysis of structure/activity of the type QSAR.

A system according to the invention includes also one or several Resource Units (061) which are connected to the computer system described above, such as for example one or several DNA chips or one or several protein chips.

In a particular mode of implementation of the method defined above, indicated (I), the Resource Units (061) include at least one means for perturbing the cell culture (CELL), by putting it in the presence of an arbitrary compound S under conditions of concentration C1 and duration T1 given by the user, the system aiming at evaluating the perturbation induced in the cell culture by the compound S under the said conditions of concentration C1 and duration T1.

By “perturbation”, one understands according to the invention a variation of the value of a state parameter of at least one marker Gi, compared to the value of the said state parameter in an ensemble of cells of reference, for example in the absence of the said arbitrary compound.

Alternatively, in a mode of implementation of the method defined above, indicated (II), the cell culture (CELL) is perturbed beforehand by a arbitrary compound S under conditions of concentration C1 and duration T1 given by the user, the said cell culture allowing the evaluation of the perturbation induced on the cell culture by the compound S under the said conditions of concentration C1 and duration T1.

In certain modes of realization of the invention described previously, the general method of determination of the state of a cell ensemble can be applied to test the effect of a compound S on a cell ensemble, for example on a given cell line, or on a plurality of cellular ensembles, for example on a plurality of distinct cell lines.

In these particular modes of realization of the invention, for which the determination of the state of an ensemble of cells is carried out for (i) a plurality of cell sets (CELL1, CELL2, . . . , CELLn), possibly (ii) a plurality of concentrations (C1, C2, . . . , Cn) of a compound S, possibly (iii) a plurality of exposure times (T1, T2, . . . , Tn) of the cells to the said compound S and if necessary (iv) a plurality of substances (S1, S2, . . . , Sn) to be tested, one can use a plurality of systems of determination of the state of at least one ensemble of prokaryotic or eukaryotic cells such as defined above, in particular if the test is performed by carrying out in a simultaneous way a plurality of executions of the general method of the invention, each execution of the general method being realized for a specific combination of (i) ensemble of prokaryotic or eukaryotic cells, (ii) concentration of compound S, (iii) exposure time of the cells to the compound S and (iv) compound S.

Thus, the present invention has also as an aim a system allowing to test the effect of a compound S on an ensemble CELL of prokaryotic or eukaryotic cells, the said system including a plurality of systems (Sys1, Sys2, . . . , Sys2) according to the invention, each one of the said systems according to the invention being adapted to determine the state of an ensemble of prokcaryotic or eukaryotic cells for a combination from at least two of the following parameters:
(I) a given ensemble CELL of prokaryotic or eukaryotic cells;
(II) a given concentration C of a compound S to be tested;
(III) a given exposure time T of the cells to a compound S to be tested; and
(iv) a given compound S.

Preferably, the device defined above allows comparative evaluations based on multiple evaluations, characterized in that the said evaluations are carried out by N systems (Sys1 . . . SysN) such as those noted (I) or (II) above, each system Sys(i), 1<=i<=N evaluating an ensemble of cells CELL perturbed by a compound S under conditions of concentration C1(i) and durations T1(i) given by the user and specific to the system Sys(i), and at least one of the systems, which is noted SysRef in the following, evaluating a undisturbed ensemble of cell used as a reference, in order to obtain by comparison with the results obtained for each system (Sys1, Sys2, . . . , SysN) an evaluation of the perturbation induced by the said compound S according to the used concentrations C1(i) (1<=i<=N) and durations T1(i) (1<=i<=N).

Alternatively, in a certain mode of implementation of the method above, indicated (III), the Resource Units (061) include at least one means for generating, starting from the cell culture (CELL), an ensemble of cells CELLtest1 obtained by putting an arbitrary amount of CELL in the presence of an arbitrary compound S1 under conditions of concentration C1 and duration T1 given by the user, and at least one means for generating, starting from the cell culture (CELL), a culture of CELLtest2 cells obtained by putting an arbitrary amount of CELL in the presence of an arbitrary compound S2 under conditions of concentration C2 and of duration T2 given by the user, then to carry out an evaluation, for a marker Gi contained in (051), of the perturbed ensembles of cells CELLtest1 and CELLtest2, allowing the Resource Unit to generate for the marker Gi one or several state parameters relating to the difference in behaviour between the cell culture CELL perturbed by S1 according to C1 and T1 and the cell culture CELL perturbed by S2 according to C2 and T2.

By extension of what precedes immediately, in a certain mode of realization of the method, the Resource Units as described in the said system (III) can generate one or several state parameters for a marker Gi contained in (051) related to the difference in behaviour, between the cell culture CELL perturbed with S1 according to C1 and T1 and the unperturbed cell culture CELL.

Thus, according to certain modes of realization of a system or a device of the invention, such as defined in the present description, the said system or the said device can be also characterized in that the Resource Units (061) include at least one means allowing to generate, starting from a cell culture CELL, a cell culture CELLtest1 obtained by putting an arbitrary amount of CELL in the presence of an arbitrary compound S1 under conditions of concentration C1 and duration T1 given by the user, in order to carry out in parallel an evaluation, for a marker Gi contained in (051), of the cultures of CELLtest1 and unperturbed CELL thus allowing the Resource Unit to produce one or several state parameters for the Gi marker related to the difference in behaviour, relative to Gi, between the cell culture CELL perturbed with S1 according to C1 and T1 and the unperturbed cell culture CELL. Means for generating from a cell culture other cell cultures exposed to given environmental conditions are currently available in the state of the art. These means can in particular consist of programmable laboratory automats, which are commercially available.

Preferably, the device described in a general way above, constitutes a means of comparative evaluations, noted ScompCT, based on multiple evaluations where each evaluation corresponds to an execution of the general method of the invention, allowing to quantify the impact of an ensemble of N conditions of concentration and exposure duration (Ci, Ti), 1<=i<=N on the perturbation of the cell ensemble CELL by a substance S, compared to a reference condition of concentration and duration (Cref, Tref) for the same substance S, ScompCT including:
a system Sys1 as defined according to the notations (I), (II) or (III) above using a set of markers Gset stored in the means (051), allowing the evaluation of an ensemble of cells CELL exposed to substance S under conditions of concentration and duration (Cref, Tref, thereby providing an reference evaluation E of the perturbation induced by the said substance S as well as a chronology T relating to the sequence of the tests of the markers Gset performed during the evaluation E, on the one hand, and
a system Sys2 as defined according to the notations (I), (II) or (III) above using the aforementioned set of markers Gset stored in (051); the means (070) containing initially the priorities of markers in Gset such as those initial priorities reproduce a global order of the markers in Gset identical to the one induces by the chronological order T obtained by Sys1 for the markers of Gset; the means (020) of Sys2 being charged with a null metric, so that the scheduling inherent to the chronology T is reproduced by Sys2 during the evaluation of the ensemble of cells CELL disturbed by the substance S under conditions of concentration and exposure durations chosen in (Ci, Ti), with 1<=i<=N.

The system for comparative evaluations ScompCT uses only once the system Sys1 then as many times as necessary the system Sys2 to measure the impact of the variations of concentrations and duration (Ci, Ti), 1<=<i<=N compared to (Cref, Tref) on the perturbation induced in the ensemble of cells CELL by the substance S, by comparison between the reference evaluation E obtained by Sys1 and the evaluations obtained by the occurrences of Sys2.

The invention also has as an aim a device of comparative evaluations using an ensemble of markers Gset allowing to quantify the impact of an ensemble of N conditions of concentration and duration (Ci, Ti), 1<=i<=N in the disturbance of an ensemble of cells CELL by a substance S, compared to reference conditions of concentration and duration (Cref, Tref) (Cref may be null) for the same substance S, characterized in that it includes:
at least N systems (Sys1, Sys2, . . . . SysN) as previously defined, each system Sys(i), 1<=i<=N using the set of markers Gset, to evaluate the ensemble of cells CELL exposed to S under conditions of concentration Ci and duration Ti given by the user and specific to the system Sys(i),
at least one system SysRef as definite previously and using the set of markers Gset to evaluate the ensemble of cells CELL exposed to S with the conditions of concentrations (Cref, Tref); knowing that if Cref=0, then SysRef assesses the unexposed ensemble of cells CELL.

The device of comparative evaluations allows to obtain, by comparing the results collected for each system, an estimation of the perturbation induced by exposure to substance S following concentrations C(i) (1<=i<=N) and Cref and durations T (I) (1<=i<=N) and Tref.

Alternatively, the system defined in a general way above, constitutes a system of comparative evaluations based on multiple evaluations, noted ScompSub, which allows to quantify, for given conditions of concentration Cref and a duration Tref, the perturbation induced by an ensemble of N substances Sub1 . . . . SubN with reference to a substance of reference Sref; ScompSub includes:
a system Sys1 as defined according to the notations (I), (II) or (III) above, using an ensemble of markers Gset stored in (051), allowing the evaluation of an ensemble of cells CELL exposed to substance Sref under conditions of concentration and duration (Cref, Tref), providing a reference evaluation E of the perturbation induced by the said substance Sref, and a chronology T of the sequence of the tests of markers Gset realized in E on one hand, and
a Sys2 system as defined according to the notations (I), (II) or (III) above, using the markers of Gset stored in (051); where means (070) initially contains priorities of the markers of Gset, so that the initial priorities reproduce a total order of the markers of Gset identical to the one induced by the chronological order T obtained by Sys1 for the markers of Gset; the means (020) of Sys2 being loaded with a null metric, so that the schedule inherent to the chronology T is reproduced by Sys2 during the evaluation of the said ensemble of cells CELL disturbed by a substance chosen in {Sub(i)}, 1<=i<=N, under the conditions of concentration and duration (Cref, Tref),

The system of comparative evaluations ScomSub uses only once the system Sys1 then, as many times as necessary the system Sys2 to measure the impact of the variations of substance (Sub(i)), 1<=<i<=N compared to Sref, at fixed condition of concentration and exposure duration (Cref, Tref), on the ensemble of cells CELL, by comparison between the evaluation of reference E and the evaluations obtained by the occurrences of Sys2.

Thus, the invention also covers a system of comparative evaluations that uses a set of markers Gset to evaluate the perturbations induced on an ensemble of cells CELL by an ensemble of N substances Sub(i), 1<=i<=N, under given conditions of concentration Cref and an exposure duration Tref, the said system comparing perturbations induced by each substance Sub(i), 1<=i<=N, to the perturbations induced, under identical conditions, by (a possibly undefined) reference substance Sref, characterized in that it includes:
at least N systems (Sys1 . . . . SysN) such as previously defined, each system Sys(i), 1<=i<=N using the set of markers Gset to evaluate the ensemble of cells (CELL) exposed to substance Sub(i) under the conditions of concentration Cref and exposure duration Tref,
at least one system SysRef such as previously defined using the set of markers Gset to evaluate the ensemble of cells (CELL) exposed to Sref under the conditions of concentrations Cref and exposure duration Tref; knowing that if Sref is undefined, then SysRef evaluates the ensemble of unexposed cells CELL.

The system of comparative evaluations allows to obtain, by comparison of the results obtained for both systems, an estimation of the variation of behaviour between the said substances Sub (i), 1<=i<=N and Sref at concentration Cref and exposure duration Tref.

The invention also has as an aim a device of comparative evaluations of the type above, using an ensemble of markers Gset, a reference system of evaluation of SysRef and N systems of evaluations Sys(i), 1<=i<=N, characterized in that:
the reference system of evaluation SysRef is executed initially to provide an evaluation of reference E as well as a chronology T concerning the sequence of the tests of markers Gset realized within the frame of the evaluation E, on one hand, and
for each system of evaluation Sys(i) the means (070) is initially configured with initial priorities for markers present in Gset, the initial priorities reproducing a global order of the markers of Gset identical to the one induced by the chronological order T obtained by SysRef for the markers of Gset; the means (020) of Sys(i) being loaded with a null metric, so that the chronology contained in T is reproduced by Sys(i) during its execution.

In certain particular modes of realization, a system or a plurality of systems according to the invention, using is characterized in that it uses an ensemble of markers M; the system or plurality of systems including a means allowing the user to specify a real, positive or null scalar value of acceptance ACCEPT which can possibly be dynamically, so that the system or plurality of systems can discriminate, at any time of the evaluation, the ensemble of markers not yet tested in two sub-sets:
the sub-set of SIGNIFICANT markers, defined as the ensemble of the markers G of M for which the priority, established in the means (070) at this moment, is greater numerically than ACCEPT value, and
the sub-set of the NONSIGNIFICANT markers, defined as the ensemble of the markers G of M for which the priority, established in the means (070) at this moment, is lower numerically than ACCEPT value.

In a highly preferred application, a system noted (I), (II) or (III) above takes into account the results of an ensemble of N evaluations {E1 . . . EN}, each evaluation Ei (1<=i<=N) having been obtained by means of a system Si; the system Si including:
an ensemble of cell CELL(i) exposed to a substance SUB(i),
an ensemble of markers Gi, producing through evaluation Ei the set of the data contained in the means (050) of Si, the said system allowing to define, for a sub-set M of markers of UNION(Gi), (1<=i<=N), UNION representing the set union operator, a weighting POND_M of the markers of M, the weight of each marker G of M in POND_M taking account of the values RELEVANCE(G), Result(G) and Iter(G) obtained in the means (051) and (052) bound the evaluations Ei (1<=i<=N) in which G took part, the weight of each marker G of M in POND_M giving an account of the total relevance of the marker G in the N evaluations {E1 . . . EN}.

Preferably, the system defined immediately above includes an ensemble of markers M containing the markers Gi having, or having not, participated in one or several previous evaluations, so that the weighting POND_M of all or part of the markers of M can be established by the system in according to the description given immediately above, the information derived from this weighting allowing to establish the data of initial priorities of the markers of M in the means (070) of the said system, the initial priority of each marker Gi of M being proportional to the weight of the marker Gi in weighting POND_M.

Alternatively, the system defined above includes an ensemble of markers M containing the markers Gi having, or having not, participated in one or several previous evaluations, so that the weighting POND_M of all or part of the markers of M can be established by the system according to the description given immediately above, the information derived from this weighting allowing to establish the metric data for relations between markers of M in the means (020) of the said system, the value of metric between two unspecified markers Gi and Gj of M being obtained by comparing of the weights of the markers Gi and Gj obtained in POND_M.

The invention thus also relates to a self-adaptation system, determining, by successive evaluations, the priority markers as well as the metric data between markers for an arbitrary ensemble M of markers Gi according to procedures outlined immediately above.

The system defined in a general way above can include a means allowing the user to specify a real positive or null scalar value of acceptance ACCEPT, which can be dynamically adjusted, so that the said system can separate at any time of the evaluation the ensemble of the markers M not yet tested in two sub-sets, respectively:
the sub-set of the SIGNIFICANT markers, defined as the ensemble of markers G of M for which the priority established in the means (070) at this moment, is greater numerically than the value ACCEPT, and
the sub-set of the NONSIGNIFICANT markers, defined as the ensemble of markers G of M for which the priority established in the means (070) at this moment, is lower numerically than the value ACCEPT.

Another purpose of the invention consists of a dynamic partitioning system (1000) known as Monitor of Cluster, allowing the dynamic assignment of Resource Units among multiple systems of evaluation, characterized in that the said Monitor of Cluster controls the dynamic partitioning of an ensemble R of a number RU(t) of similar Resource Units (061), where the number RU(t) may vary dynamically by addition and removal of Resource Units, among a number N(t) (where N(t) can vary dynamically) of systems P(i) (1<=i<=N(t)) as defined immediately above. Each systems P(i) carries out independently or not an evaluation; all evaluations being carried out in parallel and concurrently on the ensemble R. The dynamic partitioning system monitors the dynamic partition in order to guarantee that at any time each of the N(t) evaluations has access, in a relative way, to a sufficient number of Resource Units according to its immediate needs. The dynamic partitioning system uses a real scalar parameter FRACTION such as 0<=FRACTION<=1, the value of FRACTION being arbitrarily fixed by the user at any time; so that at every moment T each evaluation P(i) (1<=i<=N(T)) is allotted by the system a minimum number of Resource Units MIN(i)(T)=FRACTION*RU(T)/N(T); augmented by a variable number DYN(i)(T) of Resource Units which depends directly on the number SG(i)(T) of markers present in the sub-set of the SIGNIFICANT markers present in evaluation P(i) at the moment T, calculated according to the following formula:

DYN(i)(T)=(1−FRACTION)*RU(T)*SG(i)(T)/(ΣSG(j))(T), 1<=j<=N(T))

with the following rule:
if the number of significant markers of the process P(j), 1<=j<=N(T) is null at the moment T, then SG(j)(T)=1,
(Σ representing the addition operator),
   Then, the dynamic partitioning system assigns at any time T a number MIN(i)(T)+DYN(i)(T) to the process of evaluation P(i) (1<=i<=N(T)).
   Alternatively the dynamic partitioning system (1000) known as Monitor of Cluster monitors the dynamic partitioning of an ensemble R of a number RU(t) of similar Resource Units (061); where the number RU(t) can vary dynamically by addition or removal of Resource Units, among a number N(t) (where N(t) may vary dynamically) of systems P(i) (1<=i<=N (T)) as defined immediately is above. Each system P(i) (1<=i<=N (T)) carries out independently or not an evaluation; all evaluations being carried out in parallel and concurrently on the ensemble R. Each system P(i) (1<=i<=N(T)) uses an ensemble of a number C(i) of arbitrary markers.

The dynamic partitioning system monitors the dynamic partition in order to guarantee that at any time each of the N(t) evaluations has access, in a relative way, to a sufficient number of Resource Units according to its immediate needs. The dynamic partitioning system uses a real scalar parameter FRACTION such as 0<=FRACTION<=1, the value of FRACTION being arbitrarily fixed by the user at any time; so that at every moment T each evaluation P(i) (1<=i<=N(T)) is allotted by the system a minimum number of Resource Units:

MIN(i)(T)=FRACTION*RU(T)*C(i)/(ΣC(j), 1<=j<=N(T)),

Σ representing the operator sum, augmented by a variable number DYN(i)(T) of Resource Units which directly depends on the number SG(i)(T) of markers present in the sub-set of the SIGNIFICANT markers present in the process of evaluation P(i) at the moment T, calculated according to the following formula:

DYN(i)(T)=(1−FRACTION)*RU(T)*SG(i)(T)*C(i)/(ΣC(i)*SG(j)(T), 1<=j<=N(T))

(Σ representing the operator sum),
with the following rule:
if the number of significant markers of the process Pj (1<=j<=N(T)) is null at the moment T, then SG(j)(T)=1,
   Then, the dynamic partitioning system (1000) assigns at any time T a number MIN(i)(T)+DYN(i)(T) to the process of evaluation P(i) (1<=i<=N(T)).

The present invention is moreover illustrated with, but not limited to, the following examples.

### EXAMPLES

### Example 1

### Description of a Mode of Realization of a System According to the Invention

One wishes to carry out the evaluation of the toxicity of a collection of (15) pesticides using a toxicogenomic analysis of the impact of these pesticides on neuronal cells, according to 4 modes of exposure of the cells to the compounds:

Expo11=concentration of the C1 compound=IC50, Duration T1=24 h.
Expo12=concentration of the C1 compound=IC50, Duration T2=48 h.
Expo21=concentration of the C2 compound=IC50/10, Duration T1=24 h.
Expo22=concentration of the C2 compound=IC50/10, Duration T2=48 h.

To do so, one uses 51 biological markers of gene type in a system according to the present invention. The 15 tested pesticides are: Abamectin, Aldicarb, Aldrin, Carbaryl, Chlorpyriphos, Dicofol, Fenazaquin, Fipronil, Heptachlor, Lindan, Methoxychlor, Paraquat, Permethrine, Phosmet, Rotenone.

The 51 genes used are otherwise known to induce various pathological answers, classified in our example in 6 distinct families:

Stress, DNA Damage, Cellular Cycle, Neurotoxicity, Hormonal Response and Conformational Attack (proteins).

One is thus interested in the impact of the pesticides on these 6 families of genes. The 51 genes used are classified in these 6 families, as described further.

The system according to the invention consists of a PC computer carrying out means described in the invention, the PC being connected by a serial link to a single resource unit RU1.

RU1 contains:

DNA chips built specifically to test the expression level of the 51 genes in cells under study, each chip being configured only for one gene in our case, each chip allowing to carry out for each gene a redundant number of measurements (16) in order to stabilize the levels of expression by analysing the results on average. The spots on the chips are numbered sp1 to sp16.

A device D1 allowing to select one of the chips with DNA already characterized for a given gene and to put it in the presence of 2 biological samples E1 and E2, E1 being placed in spots sp1 to sp8, and E2 in spots sp9 to sp16.

A device D2 allowing the introduction of a cell culture C into the Resource Unit.

A device D3 allowing the introduction of a compound S into the Resource Unit.

A communication means C1 with the PC allowing the PC to specify a gene G, a concentration Co and a duration T, as well as a signal of configuration of the Resource Unit with G, Co and T,

A communication means C2 with the PC allowing the PC to ask for the execution of a test after a configuration,

A communication means C3 allowing the Resource Unit to transmit to the PC the result RES of a test,

A communication means C4 with the PC allowing the PC to ask the re-initialization of the Resource Unit

An automated device D4 allowing, on signal of C1 configuration coming from the PC, to take a sample of the cell culture C, to put it in the presence of the substance S under the condition of concentration Co and exposure T specified in the configuration.

An automated device D5 allowing, on signal of execution of the C2 test, to take a E1 sample of the cell culture C (non exposed) and a E2 sample of the cell culture C exposed to the compound by the device described previously under the conditions specified with the configuration, then to use the D1 device to select a DNA chip corresponding to the gene specified at the time of the configuration, and to put the samples E1 and E2 thus obtained in the presence of the aforementioned DNA chip according to the methods described in the D1 device,

A D6 device allowing a reading of the DNA chip in the course of tests by the D5 device, allowing to calculate a value of gene expression for the cell culture C impacted by the compound S under the conditions of configuration, the calculation of the value being done by dividing the average of the values obtained for the spot sp9 to sp16 by the average of the values obtained for the spots sp1 to sp8, the D6 device using C3 to transmit this result value to the PC,

A D7 device allowing to clean the internal components of the Resource Unit on reception of the C4 signal.

The Resource Unit thus offers testing capabilities which are a priori independent of the markers, even of the cell culture and of the compound to be tested.

According to the invention, the PC can require the configuration of the Resource Unit for a given gene, under precise conditions of concentration and exposition duration for an arbitrary cell culture exposed to an arbitrary compound.

The result of a test, RES, transmitted by the Resource Unit to the PC, is a raw numerical value which indicates the expression level of the gene indicated in the configuration, for the cell exposed to the compound under the conditions of concentration and exposure duration specified in the configuration, compared to the expression level of the same gene in an unexposed cell of identical nature. Thus, a result value of 2 indicates that the gene is 2 times more expressed in the exposed cell than in the unexposed one, a result of 0.5 indicates that the gene is 2 times less expressed in the exposed cell than in the unexposed one etc. . . .

Descriptions of 51 genes are initially placed in the means (010).

TABLE 1
IDENTITY AND CLASSIFICATION OF THE 51 GENES
PRESENT IN (010)

| | | |
|---|---|---|
| Stress | 1 | GSS |
| Stress | 2 | GPX1 |
| Stress | 3 | GSTM3 |
| Stress | 4 | SOD1 |
| Stress | 5 | TRPM2 |
| Stress | 6 | PTGS2 |
| Stress | 7 | HSPA9B |
| Stress | 8 | EPHX1 |
| Stress | 9 | NOS2A |
| DNA Damage | 10 | CDC25C |
| DNA Damage | 11 | CDKN1A |
| DNA Damage | 12 | CDK4 |
| DNA Damage | 13 | APAF1 |
| DNA Damage | 14 | ATM |
| DNA Damage | 15 | BAX |
| DNA Damage | 16 | NFKB1 |
| DNA Damage | 17 | RAD50 |
| DNA Damage | 18 | RAD51 |
| Cell cycle | 19 | FOS |
| Cell cycle | 20 | JUN |
| Cell cycle | 21 | BCL2 |
| Cell cycle | 22 | GADD45A |
| Cell cycle | 23 | MDM2 |
| Cell cycle | 24 | TP53 |
| Cell cycle | 25 | EGF |
| Cell cycle | 26 | PPARA |
| Cell cycle | 27 | TUBA1 |
| Neurotoxicity | 28 | AITCH |
| Neurotoxicity | 29 | CTSB |
| Neurotoxicity | 30 | DRD2 |
| Neurotoxicity | 31 | BZRP |
| Neurotoxicity | 32 | TH |
| Neurotoxicity | 33 | THBS1 |
| Neurotoxicity | 34 | HOXD1 |
| Neurotoxicity | 35 | ROBO1 |
| Hormonal response | 36 | TF1 |
| Hormonal response | 37 | CTSD |
| Hormonal response | 38 | PGR |
| Hormonal response | 39 | RAN |
| Hormonal response | 40 | AR |
| Hormonal response | 41 | CREB1 |
| Hormonal response | 42 | ESR1 |
| Hormonal response | 43 | CALR |
| Hormonal response | 44 | CYP19A1 |
| Hormonal response | 45 | ALB |
| Conformational attack | 46 | HSPA5 |
| Conformational attack | 47 | XBP1 |
| Conformational attack | 48 | ATF6 |
| Conformational attack | 49 | ERN1 |
| Conformational attack | 50 | C120RF8 |
| Conformational attack | 51 | A2M |

### Example 2

### Description of a Mode of Implementation of the Method According to the Invention

Having defined in example 1 the environment of the evaluation and its objectives, one now presents various modes of use of the method according to the invention, in which the behaviour of the system is influenced by the initialization of each step (evaluation of each pesticide) by the configuration of the means (020) and (070).

Each pesticide is evaluated by an distinct execution of the method. Before each evaluation, the means (010) contains the description of the markers. Each evaluation concerns one of the 15 compounds under conditions of concentration and precise durations.

The evaluation of the compounds is set under the following order: Abamectin, Aldicarb, Aldrin, Carbaryl, Chlorpyriphos, Dicofol, Fenazaquin, Fipronil, Heptachlor, Lindan, Methoxychlor, Permethrine, Phosmet, Rotenone, Paraquat.

For each compound, the conditions Expo11, Expo12, Expo21, Expo22 are tested, which overall requires 15*4=60 complete evaluations.

The 3 strategies presented thereafter are purely illustrative and do not limit in any case the invention to their specificities.

### 1 Strategy of Random Selection of the Markers:

To implement this strategy, one loads in the PC the means (030) with a model with relevance PNUL, defined by: whatever the result of a test coming from RU1 for a gene G under precise conditions of exposure, PNUL (G)=0.

Whatever the metric used in the means (020), whatever the markers “seeds” specified initially in the means (070), PNUL allow the evaluation to test at the beginning of the “seed” markers, then the evaluation adopts a random mode of selection for the other markers.

### 2 Strategy of Selection of the Markers by Class:

This strategy consists in trying to test, in priority, the markers of one of the 6 classes having a relevant level of expression regarding the evaluation. The relevant level of expression is obtained when the compound induces, for precise conditions of exposure, a gene expression with at least a factor 2 (times more or times less) in the cell exposed compared to the unexposed one. One tries moreover to impose to the system an initial random exploration, where markers are randomly tested until the system selects one of a certain class which produces relevant results: in this case, one wishes that the system preferentially studies the other markers of the class before continuing its random exploration towards one second relevant class, and so on until exhaustion of the markers.

To implement this strategy, one loads in the PC a means (070) initialized with null values of priority for all the markers (no markers “seeds”), a metric model in the means (020) defined by a numerical function MET_CLASS:

Whatever (Gi, Gj) in all 51 markers,
if Gi and Gj belong to the same class, MET_CLASS (Gi, Gj)=1;

### MET_CLASS (Gi, Gj)=0 if not.

MET_CLASS thus defines the values of the 51*50/2 relations between pairs of markers.
Lastly, one initializes the means (030) with a model of relevance PERT_CLASS: Whatever the result RES of a test coming from RU1 for a gene G under precise conditions of exposure,

### PERT_CLASS (G)=0 if 0.5<LMBO<2;

### PERT_CLASS (G)=RES if 2<=RES;

### PERT_CLASS (G)=1/RES if not.

### 3 Strategy of Self Adaptation:

This strategy aims at allowing the system to automatically exploit data from tests carried out in the past, in order to test by priority, with the help of a probabilistic approach, those markers performing best for the planed evaluation. Under given conditions of exposure of cells to a substance candidate, a marker deregulation is considered relevant, if the expression of this marker in the cells exposed to the candidate substance departs by a factor of 2 (i.e. 2 times more or 2 times less) at least from its expression in the unexposed cells. To this end, data collected from tests of a variety of substances are subjected to a statistical analysis. For each test, deregulated markers are listed. This statistical accounting is carried out for the collection of results obtained in the past for various substances and conditions of exposure, following method presented in the invention. The statistical analysis leads in our case to a symmetrical 51*51 matrix MATSTAT, which can thus be established by the system without intervention of the user.

The system is configured with a number Ns of “seed” markers. The system then chooses the markers Gi . . . GNs such that the values on the diagonal of MATSTAT are the highest Ns values (so that MATSTAT (G1, G1)>=MATSTAT (G2, G2)>= . . . >=MATSTAT (GNs, GNs)), and assigns values of priority to Gi . . . GNs in (070) large enough to avoid competition with non seeding markers during the first evaluations. Thus, in our case, the system assigns in (070) the value of priority 1000*MATSTAT (Gi, Gi) to the markers Gi (1<=i<=Ns), and the value of priority 0 to the other markers.

Moreover, the system uses the metric values present in matrix MATSTAT by application of a metric model MET_STAT configured in (020):

Whatever (Gi, Gj) in all 51 marker,

### MET_STAT (Gi, Gj)=MATSTAT (Gi, Gj)

At last, the model of relevance loaded in (030) is the model PERT_STAT:
Whatever the result RES of a test issued by RU1 for a marker G under precise conditions of exposure,

### PERT_STAT (G)=0 if 0.5<LMBO<2;

### PERT_STAT (G)=1 if not.

The results of the 3 types of strategies applied to the method according to the invention are presented hereafter. However, in order not to overload the presentation, only the results concerning the Paraquat substance are presented.

The following results are based on 3 evaluations, each using a different strategy, the target cells being neuronal cells, the mode of exposure being Expo11.

### 1 Results of the Random Strategy

The results are presented on FIG. 8.

Each time a result of test for a given marker returned a RES value lower or equal to 0.5, or higher or equal to 2, a counter is incremented (“nb.of Expressed Markers” in the graphic, FIG. 8). The figure shows the chronological evolution of this counter, time being represented by the number of unit tests carried out in sequence by RU1.

Moreover, one traces the chronological sequence of the markers tested, reported from the data present in (052), after the completion of the evaluation. This sequence must be read as proceeding from left to right. For each tested marker, if the test returned a RES value lower or equal to 0.5, or higher or equal to 2, it is marked by a cross. The results are presented on FIG. 8.

As a consequence of the random strategy introduced into the system according to the methods presented previously, the chronological order presented on FIG. 8 is random.

### 2 Results of the Strategy of Marker Selection by Class

The results are presented on FIG. 9.

Each time a result of a test returned for a given marker a RES value lower or equal to 0.5, or higher or equal to 2, a counter is incremented (“nb.of Expressed Markers” in graphics FIG. 9). The figure shows the chronological evolution of this counter, time being represented by the number of unit tests carried out in sequence by RU1.

Moreover, one traces the chronological sequence of the markers tested, reported from the data present in (052), after the completion of the evaluation. This sequence must be read as proceeding from left to right. For each tested marker, if the test returned a RES value lower or equal to 0.5, or higher or equal to 2, it is marked by a cross. The results are presented on FIG. 9.

Given the strategy of selection of the markers by class introduced into the system according to the method presented previously, the system has first tested at random a marker of the class HORMONE RESPONSE (CYP19A1), which did not provide any relevant result. Still at random, the system selected a marker of the class STRESS (SOD1) without success, then a marker of the class DNA DAMAGE (CDKN1A) for which a relevant result was found. Because of the implemented strategy, the system thus explored the whole of DNA DAMAGE genes, then started again a random selection of marker A2M (MISCONFORMATION). The relevant result of A2M led the system to explore the whole of genes of MISCONFORMATION, etc.

This mode can be easily completed by the use of seed markers selected from each gene class, so as to statistically direct by priority the evaluation towards the most relevant classes, without requiring an analysis of former results.

### 3 Results of the Strategy of Self Adaptation

The strategy of self adaptation was configured according to the methods described previously based on the results obtained for the 14 substances Abamectin, Aldicarb, Aldrin, Carbaryl, Chlorpyriphos, Dicofol, Fenazaquin, Fipronil, Heptachlor, Lindan, Methoxychlor, Permethrine, Phosmet, Rotenone, tested each according to the 4 modes of Expo11, Expo12, Expo21, Expo22 exposures. The 51 by 51 matrix MATSTAT obtained is not presented for reasons of legibility. For the evaluation of Paraquat presented here, the number of marker seeds Ns deduced by the system was fixed at 5.

The results are presented in FIG. 10.

Each time a result of test for a given marker returned a RES value lower or equal to 0.5, or higher or equal to 2, a counter is incremented (“nb.of Expressed Markers” in the graphic, FIG. 10). The figure shows the chronological evolution of this counter, time being represented by the number of unit tests carried out in sequence by RU1.

Moreover, one traces the chronological sequence of the markers tested, reported from the data present in (052), after the completion of the evaluation. This sequence must be read as proceeding from left to right. For each tested marker, if the test returned a RES value lower or equal to 0.5, or higher or equal to 2, it is marked by a cross. The results are presented in FIG. 10.

As it can be noted from the results presented in FIG. 10, the system benefited from the statistics of the former evaluations of the 14 other compounds (pesticides) to optimize the probabilities to select relevant markers by priority. The seed markers allowed to start the evaluation process, then the statistical models of metric and relevance took over, leading to an advantageous exploration of the relevant markers. This mode, although statistic, allow a premature interruption of the sequence of test by the user, thus offering substantial savings of time and means. In the specific case of the evaluation of Paraquat on neuronal cells in the mode of exposure Expo11, the strategy of self-adaptation presented here as an example makes it possible to obtain 15 relevant markers after 22 executions of RU1, whilst in the case of the example given for the marker class strategy, 35 executions of RU1 are necessary to obtain the same result.

4 Raw Results of the Evaluation of Paraquat

The results presented in table 2 show the values obtained in (052) for the evaluation of Paraquat according to the 4 modes Expo11, Expo12, Expo21, Expo22 carried out. The missing data are not relevant (ranging between 0.5 and 2 strictly).

### Example 3

### Implementation of the Method of the Invention with a Step of Analysis of Structure-Activity Relationship (QSAR) Preliminary to the Selection of the Markers Gi, at the Stage A) of the Method

The cytotoxicity of the candidate compound Rotenone was tested with the method of the invention.

The method was carried out with a preliminary step of analysis of structure-activity relationship by QSAR (for “Quantitative Structure Activity Relationship”), by comparing Rotenone with each of the 14 pesticides already tested in example 2 above.

Using QSAR analysis, three pesticides were selected as the compounds closest to Rotenone, respectively Abamectine, Carbaryl and Fenzaquin. In particular, these last three compounds have structural characteristics common with Rotenone, such as the absence of sulfur, phosphate and chlorine atoms. These three pesticides were thus selected as reference compounds, for the selection of the identity and of the order of the markers Gi to be tested for Rotenone, within the basis of 51 Gi markers described with example 1.

Thus the results obtained previously for the 51 markers during the independent evaluations of the impact of the 3 substances Abamectine, Carbaryl and Fenazaquin on the neuronal cells for the exposures Expo11, Expo12, Expo21 and Expo22 are used to build a self-adaptation matrix MATSTAT as previously described.

The tests were carried out by exposure of a neuronal cells line SH-SY5Y with a IC50/10 Rotenone concentration and an 24 hours exposure time to Rotenone.

For all the other conditions, the method is carried out as described in example 2.

At the stage A), one imposes to the system a number Ns=5 of markers seeds. The system then chooses the markers Gi . . . GNs such as the values on the diagonal of MATSTAT are the Ns highest values (so that MATSTAT (G1, G1)>=MATSTAT (G2, G2)>= . . . >=MATSTAT (GNs, GNs)), and it assigns values of priority for G1 . . . GNs in (070) large enough to avoid competition of non seeds markers during the first evaluations. Thus, in our case, the system assigns in (070) the value of priority 1000*MATSTAT (Gi, Gi) to the Gi markers (1<=i<=Ns), and the value of priority 0 to the other markers.

Moreover, the system uses the values of metrics present in matrix MATSTAT by application of a metric model MET_QSAR configured in (020):

Whatever (Gi, Gj) in all 51 markers,

### MET_QSAR (Gi, Gj)=MATSTAT (Gi, Gj)

Lastly, the model of relevance charged in (030) is model PERT_QSAR:
Whatever the RES result of a test coming from RU1 for a gene G under precise conditions of exposure,

### PERT_QSAR (G)=0 if 0.5<RES<2;

### PERT_QSAR (G)=1 if not.

Formulas and structures of the compounds which were compared, respectively Rotenone (Formula (I)), Abamectine (Formula (II)), Carbaryl (Formula (III)) and Fenzaquin (Formula (IV)) are described below.

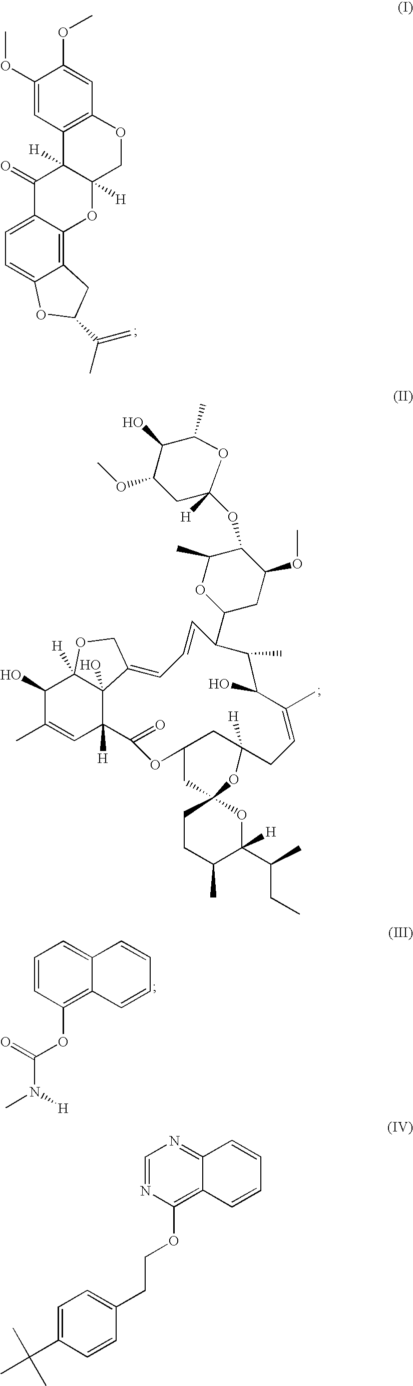

Each execution of the method corresponds to the exposure of the cells to a given concentration of Rotenone. Each cycle (unit test) in a given execution of the method corresponds to the test of a marker Gi, and more precisely, to the test of expression of a given gene (Gi marker).

After the completion of the various cycles of the execution of the method, the results generated by the various tests were analysed according to: (I) the number of informative Gi gene markers, i.e. genes which are under- or over-expressed, compared to the cells not exposed to the candidate compound, and to (II) the number of successive cycles of execution of the method.

The results are represented in FIG. 11.

Each time a result of test for a given marker returned a RES value lower or equal to 0.5, or higher or equal to 2, a counter is incremented (“nb.of Expressed Markers” in the graphic, FIG. 11). The figure shows the chronological evolution of this counter, time being represented by the number of unit tests carried out in sequence by RU1.

Moreover, one traces the chronological sequence of the markers tested, reported from the data present in (052), after the completion of the evaluation. This sequence must be read as proceeding from left to right. For each tested marker, if the test returned a RES value lower or equal to 0.5, or higher or equal to 2, it is marked by a cross. The results are presented in FIG. 11.

The results show that the number of informative Gi markers reaches a plateau after only 40 cycles of execution of the method, i.e. after only 40 Gi markers (out of 51) were tested.

The results of FIG. 11 also show that 80% of the informative markers Gi were tested after only 23 cycles of execution of the method, i.e. after only 23 Gi markers (on 51) were tested, thus allowing to characterize Rotenone by a process of classification of the compound before the 51 markers were tested (which can lead, if necessary, to a premature interruption of the evaluation by the user, if the results are considered to be sufficient).

TABLE 2

| | | | Neuron | | Neuron | |
|---|---|---|---|---|---|---|
| | | | C1 | | C2 | |
| | | | 24:00 | 48 h | 24:00 | 48 h |
| STRESS | 1 | GSS | 0.54 | 0.04 | 0.28 | 0.03 |
| | 2 | GPX1 | 0.49 | 0.02 | 0.09 | 0.02 |
| | 3 | GSTM3 | 0.71 | 0.06 | 0.36 | 0.06 |
| | 4 | SOD1 | 0.76 | 0.05 | 0.24 | 0.04 |
| | 5 | TRPM2 | | | | |
| | 6 | PTGS2 | | | | |
| | 7 | HSPA9B | 0.58 | 0.05 | 0.16 | 0.06 |
| | 8 | EPHX1 | 0.45 | 0.07 | 0.19 | 0.08 |
| | 9 | NOS2A | 0.54 | 0.04 | 0.30 | 0.06 |
| DNA DAMAGE | 10 | CDC25C | 0.62 | 0.04 | 0.37 | 0.05 |
| | 11 | CDKN1A | 0.46 | 0.02 | 0.13 | 0.02 |
| | 12 | CDK4 | 0.59 | 0.06 | 0.29 | 0.06 |
| | 13 | APAF1 | 0.47 | 0.05 | 0.20 | 0.05 |
| | 14 | ATM | 0.42 | 0.04 | 0.11 | 0.04 |
| | 15 | BAX | 0.40 | 0.05 | 0.09 | 0.05 |
| | 16 | NFKB1 | 0.54 | 0.04 | 0.26 | 0.05 |
| | 17 | RAD50 | 0.63 | 0.06 | 0.35 | 0.06 |
| | 18 | RAD51 | 0.49 | 0.05 | 0.22 | 0.06 |
| CEL CYCLE | 19 | FOS | 0.43 | 0.04 | 0.12 | 0.05 |
| | 20 | JUN | 0.61 | 0.05 | 0.33 | 0.05 |
| | 21 | BCL2 | 0.44 | 0.04 | 0.14 | 0.03 |
| | 22 | GADD45A | 0.54 | 0.04 | 0.20 | 0.04 |
| | 23 | MDM2 | 0.37 | 0.05 | 0.05 | 0.06 |
| | 24 | TP53 | 0.54 | 0.05 | 0.31 | 0.05 |
| | 25 | EGF | 0.47 | 0.04 | 0.21 | 0.05 |
| | 26 | PARA | 0.38 | 0.04 | 0.04 | 0.05 |
| | 27 | TUBA1 | 0.38 | 0.04 | 0.03 | 0.04 |
| NEUROTOXICITy | 28 | AITCH | 0.13 | 0.03 | 0.08 | 0.03 |
| | 29 | CTSB | 0.51 | 0.05 | 0.20 | 0.06 |
| | 30 | DRD2 | | | | |
| | 31 | BZRP | 0.53 | 0.04 | 0.14 | 0.04 |
| | 32 | TH | | | | |
| | 33 | THBS1 | 0.39 | 0.05 | 0.08 | 0.06 |
| | 34 | HOXD1 | 0.44 | 0.03 | 0.11 | 0.05 |
| | 35 | ROBO1 | | | | |
| HORMONAL | 36 | TF1 | 0.68 | 0.05 | 0.53 | 0.05 |
| RESPONSE | 37 | CTSD | 0.39 | 0.03 | 0.05 | 0.03 |
| | 38 | PGR | 0.42 | 0.04 | 0.12 | 0.04 |
| | 39 | RAN | | | | |
| | 40 | AR | 0.39 | 0.04 | 0.08 | 0.05 |
| | 41 | CREB1 | 0.64 | 0.03 | 0.29 | 0.05 |
| | 42 | ESR1 | | | | |
| | 43 | CALR | 0.52 | 0.04 | 0.38 | 0.11 |
| | 44 | CYP19A1 | | | | |
| | 45 | ALB | 0.57 | 0.01 | 0.34 | 0.02 |
| CONFORMATIONAL | 46 | HSPA5 | 0.55 | 0.06 | 0.33 | 0.07 |
| ATTACK | 47 | XBP1 | 0.50 | 0.05 | 0.24 | 0.06 |
| | 48 | ATF6 | 0.48 | 0.05 | 0.17 | 0.06 |
| | 49 | ERN1 | | | | |
| | 50 | C12ORF8 | 0.57 | 0.05 | 0.22 | 0.04 |
| | 51 | A2M | 0.43 | 0.03 | 0.14 | 0.04 |

1. A method for determining the state of at least one ensemble of prokaryotic or eukaryotic cells by means of the determination of the state of an ensemble of biological markers contained in, or expressed by, the said cells, the said method being implemented with a system for the determination of the state of at least an ensemble of prokaryotic or eukaryotic cells, the said system including:
1) a means (010) of data storage including an ensemble of data characterizing a number N_{M} of biological markers Gi;
2) a means (020) of data storage characterizing a metric relation R_{M} between two biological markers Gi among the N_{M} biological markers indexed in the means (010), the said means (020) including for each data of relation between two biological markers:
a reference of identification of a first biological marker G1 included in the means (010);
a reference of identification of a second biological marker G2 included in the means (010);
a value METRIC(R_{M}) defining the metric relation R_{M} between the first and the second marker;
3) a means (060-G) consisting of an ensemble of Resource Units, including a number N_{UR} of Resource Units (061), each Resource Unit (061) having for function to transmit at least one parameter of state of a biological marker Gi, contained in, or expressed by, an ensemble of cells C, towards a means (300) of evaluation of parameters of state, each Resource Unit (061) including:
a means (0611) of identification of the said Resource Unit (061);
a means (0612) of indication of the operating condition of the said Resource Unit (061) at a given moment;
a means (0613) of determination of at least one parameter of state of a biological marker Gi, contained or expressed by an ensemble of cells C;
a means (0614) of transmission of a signal between the said Resource Unit (061) and the means (300) of evaluation of parameters of state;
4) a means (050) of data storage of parameters of state of biological markers Gi, contained in, or expressed by, an ensemble of cells C, the said means (050) including:
a means (051) of data storage of relations between at least several biological markers selected in the group of the N_{M} biological markers indexed in the means (010), and storage of data of relevance, the said means (051) including, for each biological marker indexed:
a reference of identification of a first biological marker included in the means (010);
a reference of identification of a second biological marker included in the means (010);
a value METRIC (R_{M}) defining the metric of relation R_{M} between the first and the second markers; and
a value P(Gi) defining a relevance of the said first marker in a test carried out for a given marker Gi and a given cells ensemble, the said value P(Gi) being calculated by a means of analysis (400);
a means (052) of storage of results of tests carried out by one or more Resource Units (061) included in a means (060) consisting of a sub-set of the means (060-G), the said means (052) of storage including an ensemble of data of test results, each data of test result including:
a reference of identification of a biological marker included in the means (010), whose parameter of state is given by means of a Resource Unit (061) included in the means (060);
a rank value specifying the test rank of the said biological marker;
at least one value of the parameter of state defining the result of the test carried out with the said biological marker by the said Resource Unit (061).
5) a means (300) of evaluation of the execution of tests and the storage of tests results carried out by one or more Resource Units (061) included in the means (060), the said means (300) including:
a means of storage of at least one parameter of state of a biological marker Gi, the said parameter of state being generated by a Resource Unit (061);
a means of reception and transmission of a signal between the said means (300) and Resource Units (061) included in the means (060);
a means of transmission of a signal between the said means (300) and the means of control (100);
a means of transmission of a signal of the said means (300) towards a means (400) of analysis of test result.
6) a means (030) of storage of a function of computation of a value P(Gi) defining the relevance of a biological marker Gi indexed in the means (010);
7) a means (400) of analysis allowing the computation of the value P(Gi) defining the relevance of a biological marker Gi indexed in the means (010), for a given cell culture C, by using the function stored in the means (030) with at least one parameter of state defining the result of the test carried out with the said biological marker Gi by a Resource Unit (061), the said parameter of state being provided to the means (400) by the means (300), the said means (400) including a means of reception of a signal transmitted by the means (300);
8) a means (070) of Operational Planning including:
an ensemble of data for priority of execution of tests for the biological markers Gi, the said means (070) including, for each biological marker Gi:
a reference of identification of the said biological marker Gi; and
a value PRIORITY defining the priority rank of the said biological marker Gi;
a means of transmission of signals between the said means (070) and the method of control (100);
means allowing the initial configuration of the priority rank for each marker; priority data being positive or null;
9) a means (200) of initialization of the system, including:
a means of transmission of a signal between the said means (200) and the method of control (100);
a means of transmission of a signal between the said means (200) and the means (010) including an ensemble of data characterizing a number N_{M} of markers Gi;
a means of transmission of a signal between the said means (200) and the means (020) of data storage characterizing a metric relation R_{M} between two biological markers Gi.
a means of transmission of a signal between the said means (200) and the means (050) of data storage of parameters of state of biological markers Gi, contained or expressed by a cell ensemble C.
a means of transmission of a signal between the said means (200) and the means (070) including an ensemble of data characterizing the initial priorities of the N_{M} biological markers Gi.
10) a means of control (100) including:
means of transmission of a signal between the said means (100) and each Resource Unit (061);
means of transmission of a signal between the said means (100) and the means (070) of Operational Planning;
means of transmission of a signal between the said means (100) and the means (300) of analysis of the test results;
means of transmission of a signal between the said means (100) and the means (050) of data storage;
means of command of operation of the Units of Resource (061),
the said method including the following stages:
A) initialize the system according to a step during which the method of control (100) carries out the following orders:
a1)
(i) load the data contained in the means (010) and (020) into the storage means (051);
(ii) load into the means (030) the information and instructions concerning the computational function of relevance values used for the markers Gi contained in the means (051);
(iii) load into the means (070) the initial priority information, for at least part of the biological markers Gi selected among the N_{M} biological markers;
and
a2) select, starting from an ensemble (060-G) of Resource Units (061), an ensemble (060) of Resource Units (061) able to perform a test of state for at least part of the biological markers Gi referred to at the step a1) in the means (051) and (070), then initialize each Resource Unit (061) of the means (060);
B) start the system according to a step during which the method of control (100) carries out the following orders:
b1) select the marker Gi indexed in the means (070) having the highest value of priority PRIORITY, then remove the data of the said Gi marker from the contents of the means (070);
b2) add, in the storage means (052), data associated to the selected marker Gi, respectively:
the identification reference of the said biological marker Gi included in the means (051), and
a rank value specifying the test rank of the said biological marker Gi.
b3) select a Resource Unit (061) able to carry out a test of state of the said biological marker Gi and transmit to the means (300) an execution command for a test of state of the said selected marker Gi, the said test being carried out by the said Resource Unit (061) following its configuration with the marker Gi, the said Resource Unit being therefore assigned to a “BUSY” value of state;
C) recover the results of the tests carried out at the stage B), according to the following steps:
c1) load the parameters of state generated by each Resource Unit (061) selected at the end of the step B) into the storage means included in the evaluation means (300);
c2) transfer the parameter(s) of state loaded at the step c1) towards the storage means (052) and towards the analysis means (400);
D) analyze the results of the tests recovered at the stage C), according to the following steps:
d1) compute, for each biological marker Gi for which at least one parameter of state was transmitted to the means of analysis (400), the value P(Gi) defining the relevance of the said marker Gi, by using the function stored in the means (030) with the said parameter of state defining the result of the test carried out on the said biological marker Gi by a Resource Unit (061);
d2) propagate the value P(Gi) computed at the step d1) for the said biological marker Gi towards the storage means (051), the propagation consisting in assigning to each data of relation between the not yet tested markers Gj and Gi a value of propagation: PROPAGATION(Gj, Gi)=P(Gi) in the storage means (051);
d3) reinitialize the Resource Unit (061) used for the said biological marker Gi by assigning to the said Resource Unit the state value “FREE”.
E) update the marker data contained in the system, according to the following steps:
e1) carry out a new classification about the not yet tested Gi markers indexed in the storage means (051), while assigning to each not yet tested Gi marker a value of priority, the said value consisting of a value of weight Pi being function of the value of the result of the following equation (1):
Pi=ΣMETRIC(Gi,Gk)*PROPAGATION(Gi,Gk); 1<=k<=N
in which:
Σ is the operator “sum”
N is the number of markers present in the means (051);
METRIC(Gi,Gk) is the value of the metric relation between the marker Gi and the marker Gk contained in the means (051), loaded at the step a1)-(i);
PROPAGATION(Gi,Gk) is the parameter defining the value propagated at the step d2) during the test of the marker Gk if it was already tested; PROPAGATION(Gi,Gk)=0 if Gk was not yet tested; and
e2) update the data in the means (070) of Operational Planning with the new classification induced by the values of weight obtained at the step e1) for each not yet tested Gi marker.
cycles of stages B) to E) being reiterated until the first occurrence of one of the following conditions in stage F):
F) conditions of termination:
(i) interruption of the system by the user;
(ii) the value of a parameter of state for a Gi marker generated by a Resource Unit (061) was preset as an abortion condition of the system;
(iii) the stages A) to E) of the method were carried out for all the Gi markers indexed in the storage means (051),
given that the ensemble of the parameters of state contained in the storage means (052) at the end of the stage F) constitutes the state of the ensemble(s) of prokaryotic or eukaryotic cells which is determined by the method, and given that at the end of the stage F), the storage means (052) contains information about the chronological order in which the markers were tested by the method.

2. Method according to claim 1, characterized in that the biological markers Gi include one marker or a combination of markers chosen among the following markers:
the presence or the absence of a nucleic acid sequence in the genome of the tested cells, including the presence or the absence of a polynucleotide sequence coding a protein, or the presence or the absence of a polynucleotide sequence regulating the expression of a gene;
the presence or the absence of polymorphic sites in the sequence of a genomic nucleic acid of the cells tested;
the presence or the absence of a specific allele in a polymorphic site in the genomic nucleic acid of the cells tested;
the presence or the absence of a transcription product of a gene contained in the genome of the cells tested, including a messenger RNA (mRNA) or a complementary DNA (cDNA);
the presence or the absence of a small interfering RNA of the RNAi type, of the small interfering RNA of the RNAsi type, of an interfering micro-RNA of the RNAmi type, of a small nucleolar RNA of the RNAsno type, of a small nuclear RNA of the RNA Sn type;
the level of transcription of a gene contained in the genome of the cells tested;
the presence or the absence of a protein coded by the genome of the cells tested, or the presence or the absence of a protein coded by an organism hosted by the cells tested, for example a viral or fungic organism;
the level of translation of a protein coded by the genome of the cells tested, or the level of translation of a protein coded by an organism hosted by the cells tested;
the intracellular or extracellular amount of a protein coded by the genome of the cells tested, or the intracellular or extracellular amount of a protein coded by an organism hosted by the cells tested;
the presence or the absence, or the quantity, of any other detectable cellular metabolite, including transcription factors, factors of epigenetic control, hormones, cofactors of enzymes, cofactors of intracellular or extracellular receptors, lipids, fatty-acids, polyosides, vitamins or oligo-elements.

3. Method according to claim 1, characterized in that the Resource Units (061) selected at step a2) can be DNA arrays, protein chips or other “-omics” devices.

4. Method according to claim 1, characterized in that the ensemble of cells tested is a culture of cells of a given type CELL, incubated for a time T with a substance S at concentration C.

5. Method according to claim 1, characterized in that at step a1) the value PROPAGATION is set to zero for all the values P(Gi) contained in the means of storage (051).

6. Method according to claim 1, characterized in that, at the stage B) of the life cycle of the method, a given Resource Unit (061) can be selected a plurality of times, depending on its availability and its capacity to carry out the test ordered for a given marker Gi.

7. Method according to claim 1, characterized in that at the end of the stage C), the means (052) contains at least one value of parameter of state, for each marker Gi marker indexed in the means (052).

8. Method according to claim 1, characterized in that at the stage D), the value of pertinence P(Gi) for a marker Gi is calculated in accordance with the following conditions:
(I) given an ensemble of cells C; and
(II) given the execution of a Resource Unit “RessourceId1” configured with a marker Gi and the sample derived from C;
(III) then:
Pertinence(Gi)=0 if the execution of the Resource Unit “RessourceId1” does not generate any significant result, in the context of the test of state of the ensemble of cells which is the subject of the execution of the method
If not, Pertinence(Gi)>0

9. Method according to claim 1, characterized in that it includes in addition a step a) (iii-1) setting a priority rank to at least a part of the N_{M} markers Gi having their data stored in the means (010) and (020), the said step a) (iii-1) being selected among one of the following steps:
(1) a step of attribution of a priority rank to each marker Gi, this rank depending on the degree of relevance of marker Gi in the kind of cellular disturbance investigated by the execution of the method in progress.
(2) a step of attribution of a priority rank to each marker Gi, this rank depending on the degree of relevance of marker Gi considering the chemical structure of the substance S tested during the execution of the method in progress.

10. Method according to claim 9, characterized in that the step a) (iii-1) consists in a step attributing a priority rank to each Gi marker, this rank being computed based on the results of a QSAR analysis between the chemical structure of the substance S tested and the chemical structures of compounds tested in previous instances of execution of the method.

11. Method for simultaneous determination of the state of a plurality of prokaryotic or eukaryotes cell ensembles by means of the determination of the state of an ensemble of biological markers contained in, or expressed by, the cells of each cell ensemble, the said method including the following stages:
1) realize, for each cell ensemble belonging to the plurality of cell ensembles, a method according to claim 1;
2) recover, for each cell ensemble, all parameters of state contained in the means of storage (052) at the end of the stage F) carried out with the said cell ensemble, the recovered data characterizing the state of the plurality of cell ensembles determined by the method.

12. Method for testing the effect of a substance (S) on an ensemble (CELL) of prokaryotic or eukaryotes cells including the following steps:
a) determine a number of combinations of (i) the concentration of the said the substance (S) and (ii) of exposure time of the cell ensemble (CELL) to the said substance (S), to carry out the test;
b) carry out as many instances of execution of the method according to claim 1 than there are combinations determined at the step a);
c) determine the effect of the substance (S) on the ensemble of prokaryotic cells or eukaryotes cells (CELL), the said effect being determined from the totality of the state parameters of markers Gi contained in the means of storage (052) after completion, at step b), of the last execution of the method.

13. Method for testing the effect of a substance (S) on a plurality of ensembles of prokaryotic cells or eukaryotes cells including the following steps:
a) determine a number of combinations (i) of ensembles of cells contained in the said plurality of cell ensembles, (ii) of concentration of the said substance (S) and (iii) of exposure time of the cells to the said substance (S), to carry out the test;
b) carry out as many instances of execution of the method according to claim 1 than there are combinations determined at the stage a);
c) determine the effect of the substance (S) on the plurality of prokaryotic or eukaryotes cell ensembles (CELL1, CELL2, . . . , CELLn), the said effect being determined by the totality of the parameters of state of the markers Gi contained in the means of storage (052) after completion, at the stage b), of the last execution of the method.

14. A system to determine the state of at least an ensemble of prokaryotic or eukaryotic cells by means of the determination of the state of an ensemble of biological markers contained or expressed by the said cells, the said system including::
1) a means (010) of data storage including an ensemble of data characterizing a number N_{M} of biological markers Gi;
2) a means (020) of data storage characterizing a metric relation R_{M} between two biological markers Gi among the N_{M} biological markers indexed in the means (010), the said means (020) including, for each data of relation between two biological markers:
a reference of identification of a first biological marker G1 included in the means (010);
a reference of identification of a second biological marker G2 included in the means (010);
a value METRIC(R_{M}) defining metric relation R_{M} between the first and the second marker;
3) a means (060-G) consisting in an ensemble of Resource Units, including a number N_{UR} of Resource Units (061), each Resource Unit (061) having for function to transmit at least one parameter of state of a biological marker Gi, contained or expressed by a cell ensemble C, towards a means (300) of evaluation of parameters of state, each Resource Unit (061) including
a means (0611) of identification of the said Resource Unit (061);
a means (0612) of indication of the operating condition of the said Resource Unit (061) at a given time;
a means (0613) of determination of at least a parameter of state of a biological marker Gi, contained or expressed by a cell culture C;
means (0614) of transmission of a signal between the said Resource Unit (061) and the means (300) of evaluation of parameters of state;
4) a means (050) of data storage of parameters of state of biological markers Gi, contained or expressed by a cell culture C, the said means (050) including:
a means (051) of data storage of relations between at least several biological markers selected in the group of the biological N_{M} markers indexed in the means (010), and of storage of relevance data, the said means (051) including, for each biological marker indexed in this one:
a reference of identification of a first biological marker included in the means (010);
a reference of identification of a second biological marker included in the means (010);
a value METRIC(R_{M}) defining metric relation Rm between the first and the second marker; and
a value P(Gi) defining a relevance of the said first marker in a test carried out for a given Gi marker and for a given ensemble of cells, the said value P(Gi) being computed by a means (400) of analysis;
a means (052) of storage of results of tests carried out by one or more Resource Units (061) included in a means (060) consisting of a sub-set of the means (060-G), the said means (052) of storage including an ensemble of data of test results, each data of test result including:
a reference of identification of a biological marker included in the means (010) and whose parameter of state is given by means of a Resource Unit (061) included in the means (060);
a rank value specifying the test rank of the said biological marker;
at least one value of parameter of state defining the result of the test carried out with the said biological marker by the said Resource Unit (061).
5) a means (300) of evaluation of the execution of tests and the storage of results of tests carried out by one or more Resource Units (061) included in the means (060), the said means (300) including:
means of storage of at least one parameter of state of a biological marker Gi, the said parameter of state being generated by a Resource Unit (061);
means of reception and transmission of a signal between the said means (300) and Resource Units (061) included in the means (060);
a means of transmission of a signal between the said means (300) and the method of control (100);
a means of transmission of a signal of the said means (300) towards a means (400) of analysis of the results of test.
6) a means (030) for the storage of a function of computation of a value P(Gi) defining the relevance of a biological marker Gi indexed in the means (010);
7) a means (400) of analysis for the computation of the value P(Gi) defining the relevance of a biological marker Gi indexed in the means (010), for a given cell ensemble C, using the function stored in the means (030) with at least one parameter of state defining the result of the test carried out with the said biological marker Gi by a Resource Unit (061), the said parameter of state being provided to the means (400) by the means (300), the said means (400) including a means of reception of a signal transmitted by the means (300);
8) a means (070) of Operational Planning including:
an ensemble of data of priority of execution of tests for the biological markers Gi, the said means (070) including, for each biological marker Gi:
a reference of identification of the said biological marker Gi; and
a value PRIORITY defining the rank of priority of the said biological marker Gi;
means of transmission of signals between the said means (070) and the method of control (100);
means allowing the initial configuration of the data of priority for each marker; data of priority being positive or null values;
9) a means (200) of Initialization of the system, including:
means of transmission of a signal between the said means (200) and the method of control (100);
means of transmission of a signal between the said means (200) and the means (010) including an ensemble of data characterizing a number N_{M} of biological markers Gi;
means of transmission of a signal between the said means (200) and the means (020) of data storage characterizing a metric relation R_{M} between two biological markers Gi.
means of transmission of a signal between the said means (200) and the means (050) of storage of data of parameters of state of biological markers Gi, contained or expressed by a cell ensemble C.
means of transmission of a signal between the said means (200) and the means (070) including an ensemble of data characterizing the initial priorities of the N_{M} biological markers Gi.
10) a method of control (100) including:
means of transmission of a signal between the said means (100) and each Resource Unit (061);
means of transmission of a signal between the said means (100) and the means (070) of Operational Planning;
means of transmission of a signal between the said means (100) and the means (300) of analysis of the results of test;
means of transmission of a signal between the said means (100) and the means (050) of data storage;
means of command of operation of the Resource Units (061).

15. A system to determine the state of at least an ensemble of prokaryotic or eukaryotic cells according to the claim 14, characterized in that the units of resources (061) include at least a means to perturb the cell ensemble (CELL), by putting it in the presence of an arbitrary compound S under given conditions of concentration and durations which are set by the user, the system aiming at evaluating the perturbation induced in the cell ensemble by the compound S under the said conditions of concentration and duration.

16. A device to test the effect of a compound (S) on a prokaryotic or eukaryotic cell ensemble (CELL), the said device including a plurality of systems according to claim 14, each one of the said systems according to the invention being adapted to determine the state of an ensemble of prokaryotic or eukaryotic cells for a combination of at least two of the following parameters:
(I) a given ensemble of prokaryotic or eukaryotic cells;
(II) a given concentration of a compound (S) to be tested;
(III) one given exposure time of the cells to a compound (S) to be tested; and
(iv) a given compound (S).

17. Method according to claim 2, characterized in that the Resource Units (061) selected at step a2) can be DNA arrays, protein chips or other “-omics” devices.

18. Method according to claim 2, characterized in that the ensemble of cells tested is a culture of cells of a given type CELL, incubated for a time T with a substance S at concentration C.

19. Method according to claim 2, characterized in that at step a1) the value PROPAGATION is set to zero for all the values P(Gi) contained in the means of storage(051).

20. Method according to claim 2, characterized in that, at the stage B) of the life cycle of the method, a given Resource Unit (061) can be selected a plurality of times, depending on its availability and its capacity to carry out the test ordered for a given marker Gi.
